(19) United States
(12) Patent Application Publication
(10) Pub. No.: US 2024/0279608 A1
(43) Pub. Date: Aug. 22, 2024

(54) PRODUCTION PROCESSES FOR HIGHLY VIABLE, ENGRAFTABLE HUMAN CHONDROCYTES FROM STEM CELLS

(71) Applicant: University of Southern California, Los Angeles, CA (US)

(72) Inventor: Denis Evseenko, Tarzana, CA (US)

(21) Appl. No.: 18/561,253

(22) PCT Filed: May 26, 2022
(86) PCT No.: PCT/US2022/031191

§ 371 Date: Nov. 15, 2023

Related U.S. Application Data
(60) Provisional application No. 63/193,382, filed on May 26, 2021.

(51) Int. Cl. C12N 5/077

(52) U.S. Cl. CPC C12N 5/0655; C12N 2500/92; C12N 2501/105; C12N 2501/115; C12N 2501/155; C12N 2501/16; C12N 2501/415; C12N 2501/727; C12N 2506/02; C12N 2506/45; C12N 2513/00; C12N 2533/52

(57) ABSTRACT

The disclosure provides for production processes for directed differentiation of stem cells into chondrocytes in fully defined animal free conditions, and uses thereof, including for focal repair of articular cartilage.

### CROSS REFERENCE TO RELATED APPLICATIONS

This application claims priority under 35 U.S.C. § 119 from Provisional Application Serial No. 63/193,382, filed May 26, 2021, the disclosure of which is incorporated herein by reference.

### TECHNICAL FIELD

The disclosure provides for production processes for directed differentiation of stem cells into chondrocytes in fully defined animal free conditions, and uses thereof, including for focal repair of articular cartilage.

### BACKGROUND

Degeneration of articular cartilage also known as osteoarthritis is a major cause of disability in the United States affecting more than 20 Million people. There are no efficient therapies for this condition, which explains a very high percentage of patients undergoing highly invasive and expensive total joint replacement procedure. Some novel treatments using allogenic cartilage from pediatric donors (postmortem) have recently showed promising results, but supply of this donor tissue is highly restricted, expensive and carries some risk of infection.

### SUMMARY

Osteoarthritis (OA) impacts hundreds of millions of people worldwide, with those affected incurring significant physical and financial burdens. Injury to the articular surface is a major contributing risk factor for the development of OA. Current cartilage repair strategies are moderately effective at reducing pain but often replace damaged tissue with biomechanically inferior fibrocartilage. Described herein are production processes for directed differentiation of pluripotent stem cells into chondrocytes in fully defined animal-free conditions. The chondrocytes made by the processes of the disclosure exhibited long-term functional repair of porcine articular cartilage. Accordingly, the processes disclosed herein provide a new clinical paradigm for articular cartilage repair and mitigation of the associated risk of OA.

In a particular embodiment, the disclosure provides a process to produce highly viable, engraftable human chondrocytes from pluripotent stem cells, comprising: culturing stem cells on laminin coated tissue culture vessel (s) comprising a serum-free, stabilized cell culture medium for human embryonic stem (ES) or induced pluripotent stem cells (iPSCs); transferring the stem cells to a 3-D condition/spin culture system and culturing the stem cells in a medium for 5 days or more, wherein the medium comprises a serum-free, stabilized cell culture medium for human embryonic stem (ES) or induced pluripotent stem cells (iPSCs), and a ROCK inhibitor; differentiating the stem cells to chondrocyte-like cells by culturing the stem cells under the following conditions: (a) culturing the stem cells in a mesoderm induction differentiation medium for 3 days or more, wherein the mesoderm induction differentiation medium comprises a chemically defined, serum-free hematopoietic cell medium, ROCK inhibitor, Wnt3a, Activin A, and FGF₂; (b) culturing the stem cells in a MI-B day differentiation medium for 3 days or more, wherein the MI-B day differentiation medium comprises a chemically defined, serum-free hematopoietic cell medium, Wnt3a, Noggin, and FGF₂; (c) culturing the stem cells in chondrogenic induction differentiation medium A for 3 days or more, wherein the chondrogenic differentiation medium comprises a chemically defined, serum-free hematopoietic cell medium, FGF₂, and BMP4; sorting the differentiated stem cells by using magnetic cell separation to identify CD309 and CD326 negative cells, which are collected; culturing the CD309 and CD326 negative cells in chondrogenic induction differentiation medium B for seven days or more to form chondrocytes, wherein chondrogenic induction differentiation medium B comprises a chemically defined, serum-free hematopoietic cell medium, FGF2, IGF1, SHH, BMP4, ROCK inhibitor, and Primocin; culturing the chondrocytes in maintenance media for 7 days or more; wherein the maintenance media comprises a chemically defined, serum-free hematopoietic cell medium, FGF2, IGF1, LIF, TGF-β1, BMP4, ROCK and Primocin; optionally, treating the chondrocytes with an agent or compound that enhances anabolism, extracellular matrix secretion, promotes cell survival and or proliferation, reduces catabolismand/or reduces degeneration of extracellular matrix in chondrocytes; and optionally, cryopreserving the chondrocytes with collagen membranes in a defined, serum-free, and animal component-free freezing medium that comprises a ROCK inhibitor. In a further embodiment, the stem cells are human embryonic stem cells. In yet a further embodiment, the stem cells are ESI-017 GMP grade cells. In another embodiment, the tissue culture vessel is tissue culture flask (s) or CellSTACK® cull chambers. In yet another embodiment, the serum-free, stabilized cell culture medium for human embryonic stem (ES) or induced pluripotent stem cells (iPSCs) is mTeSR medium. In a further embodiment, the ROCK inhibitor is Y-27632. In yet a further embodiment, a billion or more stems cells are transferred to the 3-D conditions/spin culture conditions. In another embodiment, the a chemically defined, serum-free hematopoietic cell medium is X-Vivo medium. In yet another embodiment, the magnetic cell separation utilizes antibodies to CD326 and CD309 in a biotin/streptavidin pulldown system. In a certain embodiment, the chondrocytes are treated with an agent or compound that enhances anabolism, extracellular matrix secretion, promotes cell survival and or proliferation, reduces catabolism and/or reduces degeneration of extracellular matrix in chondrocytes, having the general structure of Formula I, II or III:

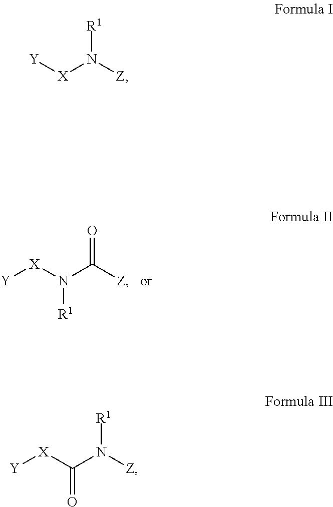

wherein,
X is

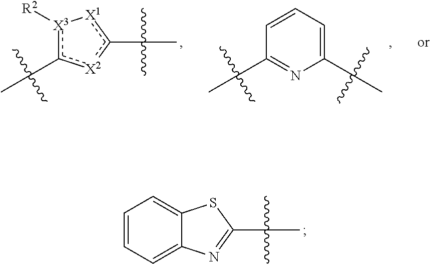

Y is

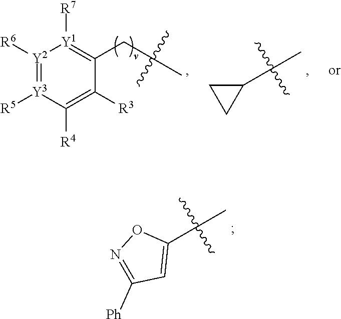

Z is

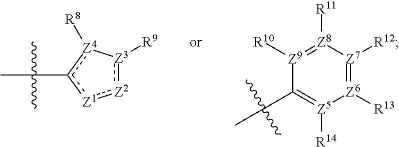

X¹ is S, CH, or NH; X² is S, CH, or N; X³ is CR² or S; Y¹ is CR⁷ or N; Y² is CR⁶ or N; Y³ is CR⁵ or N; Z¹ is O or CH; Z² is O, N, NH or CH; Z³ is CR⁹ or N; Z⁴ is CR⁸ or N; Z⁵ is N or CR¹⁴; Z⁶ is N or CR¹³; Z⁷ is N or CR¹²; Z⁸ is N or CR¹¹; Z⁹ is N or CR¹⁰; v is 0 or 1; R¹, R², R⁸ and R⁹ are independently selected from H, D, (C₁-C₃) alkyl, (C₁-C₃) alkenyl, halo, cyano, hydroxyl, nitro, thiol, and amino; R³-R⁷ and R¹⁰-R¹⁴ are independently selected from H, D, (C₁-C₃) alkyl, (C₁-C₃) alkenyl, halo, cyano, hydroxyl, nitro, thiol, amino, OC(R¹⁵)₃, OCH(R¹⁵)₂, OCH₂(R¹⁵),

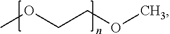

wherein n is an integer from 1 to 5; and R¹⁵ is independently H, halo, or a (C₁-C₃) alkyl; and wherein the compound modulates (i) inflammatory response and/or (ii) tissue degeneration. In a further embodiment, X is selected from the group consisting of

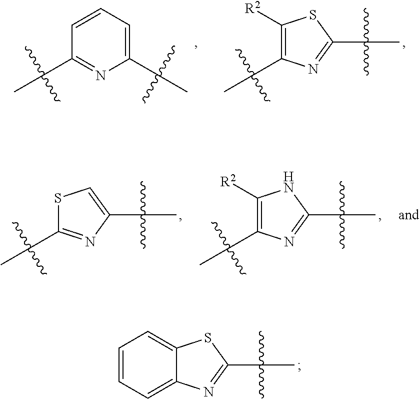

and wherein R² is an H, D, (C₁-C₃) alkyl, (C₁-C₃) alkenyl, halo, cyano, hydroxyl, nitro, thiol, or amino. In yet a further embodiment, Y is selected from the group consisting of:

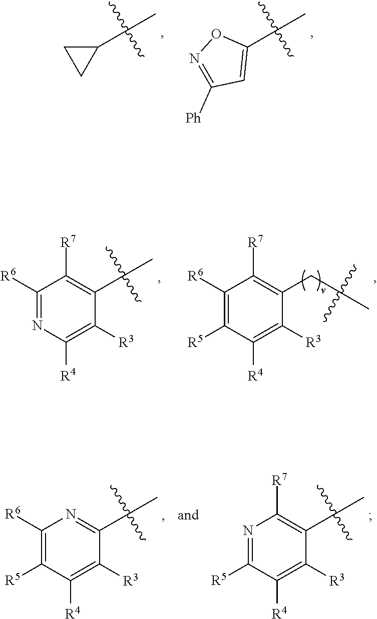

and wherein v is 0 or 1; R³-R⁷ are independently selected from H, D, (C₁-C₃) alkyl, (C₁-C₃)alkenyl, halo, cyano, hydroxyl, nitro, thiol, amino, OC(R¹⁵)₃, OCH(R¹⁵)₂, OCH₂(R¹⁵),

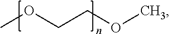

and wherein n is an integer from 1 to 5. In another embodiment, z is selected from the group consisting of:

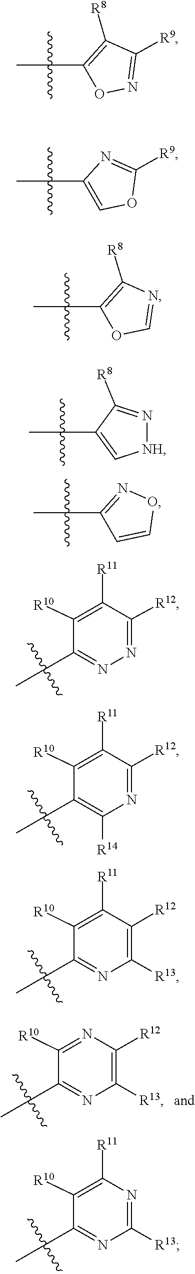

and wherein, R⁸ and R⁹ are independently selected from H, D, (C₁-C₃) alkyl, (C₁-C₃) alkenyl, halo, cyano, hydroxyl, nitro, thiol, and amino; R¹⁰-R¹⁴ are independently selected from H, D, (C₁-C₃) alkyl, (C₁-C₃) alkenyl, halo, cyano, hydroxyl, nitro, thiol, amino, OC (R¹⁵)₃, OCH (R¹⁵)₂, OCH₂(R¹⁵),

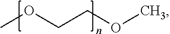

wherein n is an integer from 1 to 5; and wherein R¹⁵ is independently H, halo, or a (C₁-C₃) alkyl. In yet another embodiment, the compound has a structure selected from the group consisting of:

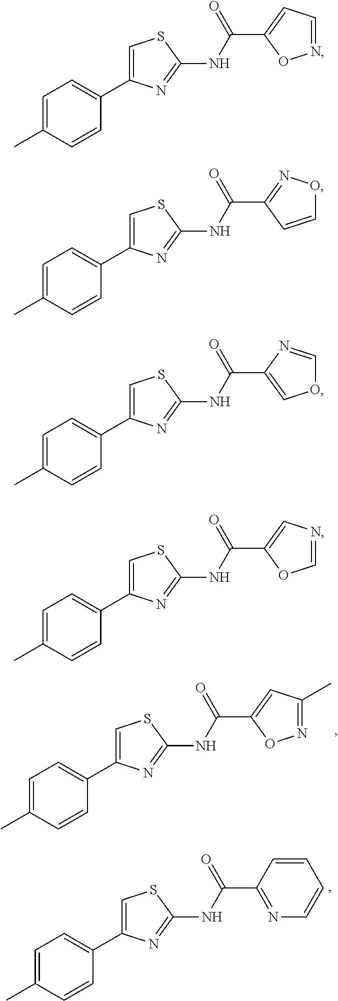

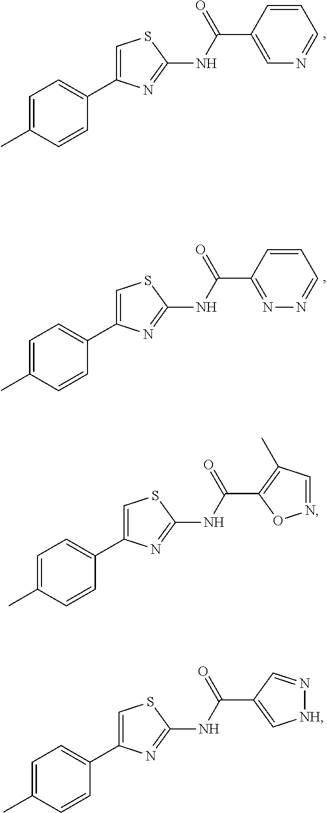 [SMILES: Cc1ccc(-c2csc(NC(=O)c3cccnc3)n2)cc1 ; Cc1ccc(-c2csc(NC(=O)c3cccnn3)n2)cc1 ; Cc1ccc(-c2csc(NC(=O)c3oncc3C)n2)cc1 ; Cc1ccc(-c2csc(NC(=O)c3cn[nH]c3)n2)cc1]

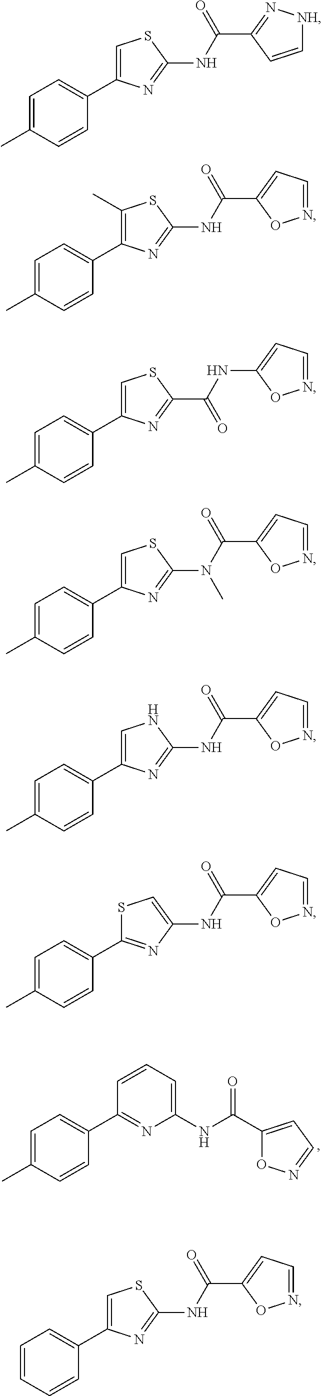

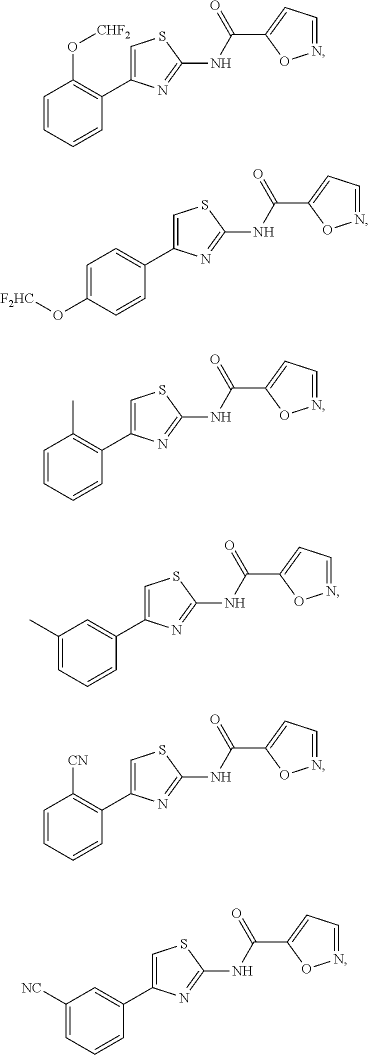 [SMILES: O=C(Nc1nc(-c2ccccc2OC(F)F)cs1)c1ccno1 ; O=C(Nc1nc(-c2ccc(OC(F)F)cc2)cs1)c1ccno1 ; Cc1ccccc1-c1csc(NC(=O)c2ccno2)n1 ; Cc1cccc(-c2csc(NC(=O)c3ccno3)n2)c1 ; N#Cc1ccccc1-c1csc(NC(=O)c2ccno2)n1 ; N#Cc1cccc(-c2csc(NC(=O)c3ccno3)n2)c1]

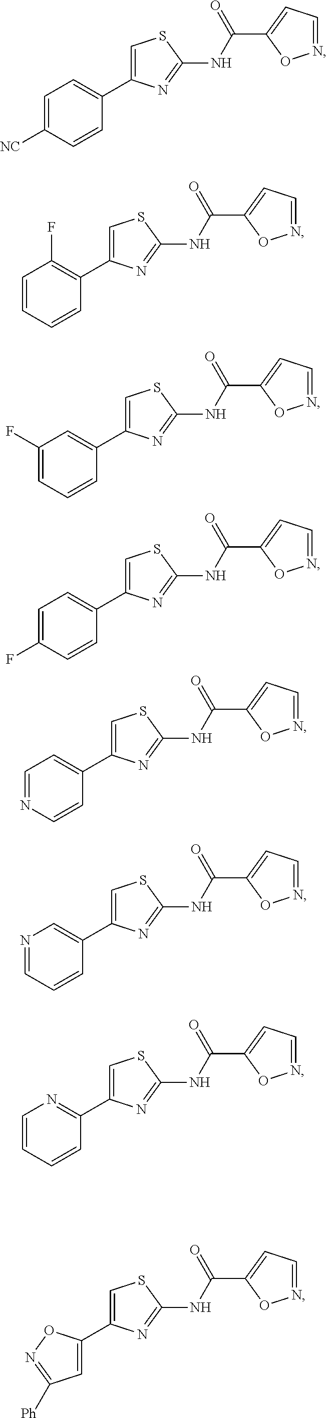

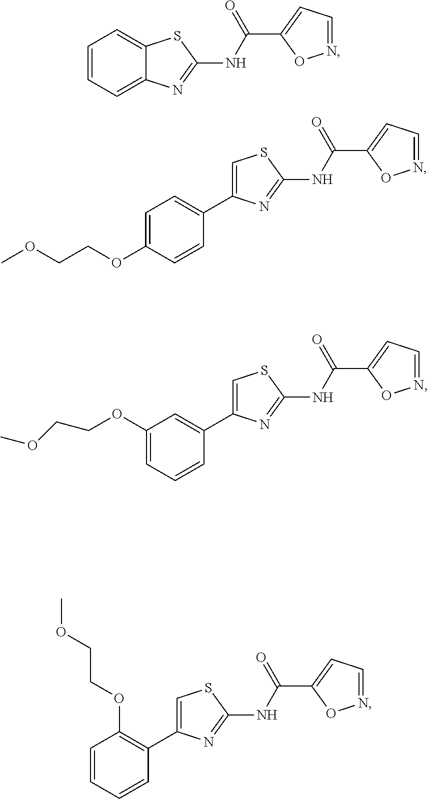 [SMILES: O=C(Nc1nc2ccccc2s1)c1ccno1 ; COCCOc1ccc(-c2csc(NC(=O)c3ccno3)n2)cc1 ; COCCOc1cccc(-c2csc(NC(=O)c3ccno3)n2)c1 ; COCCOc1ccccc1-c1csc(NC(=O)c2ccno2)n1]

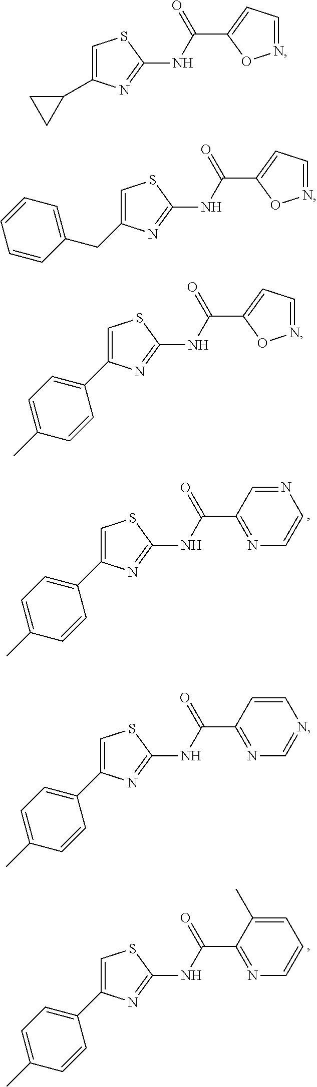 [SMILES: O=C(Nc1nc(C2CC2)cs1)c1ccno1 ; O=C(Nc1nc(Cc2ccccc2)cs1)c1ccno1 ; Cc1ccc(-c2csc(NC(=O)c3ccno3)n2)cc1 ; Cc1ccc(-c2csc(NC(=O)c3cnccn3)n2)cc1 ; Cc1ccc(-c2csc(NC(=O)c3ccncn3)n2)cc1 ; Cc1ccc(-c2csc(NC(=O)c3ncccc3C)n2)cc1]

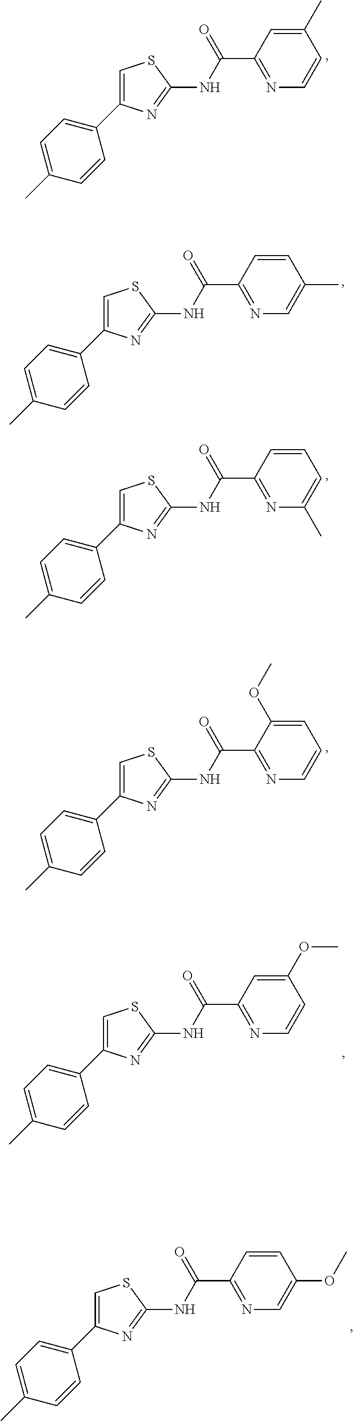

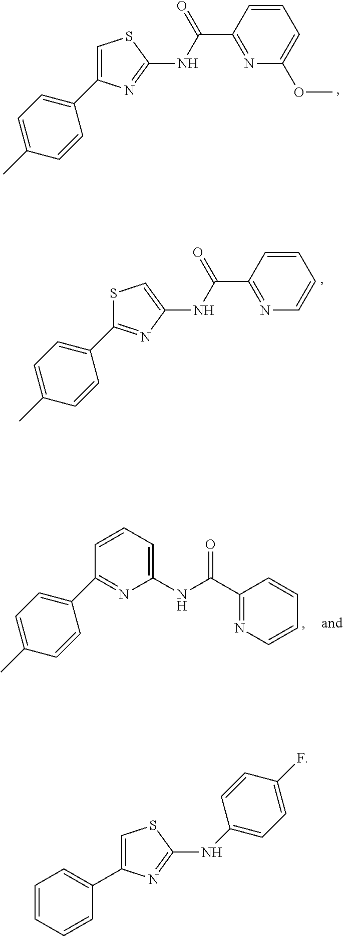

In a particular embodiment, the compound is selected from the group consisting of:

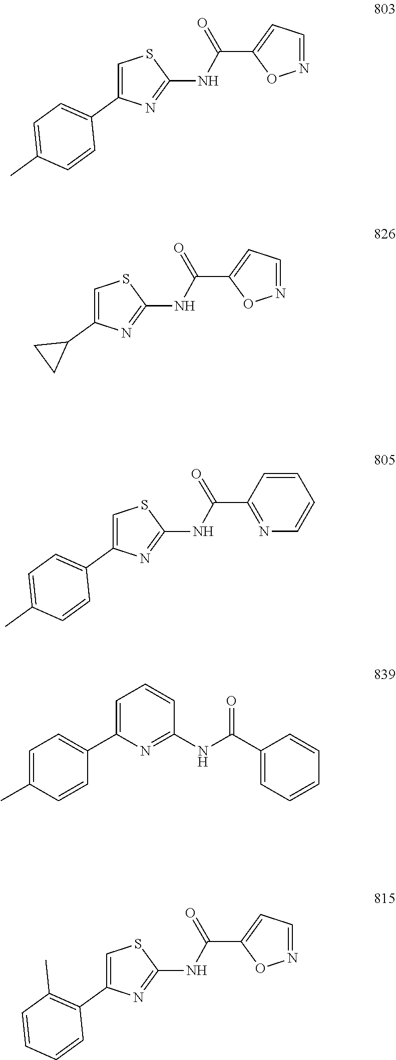 [SMILES: Cc1ccc(-c2csc(NC(=O)c3ccno3)n2)cc1 ; O=C(Nc1nc(C2CC2)cs1)c1ccno1 ; Cc1ccc(-c2csc(NC(=O)c3ccccn3)n2)cc1 ; Cc1ccc(-c2cccc(NC(=O)c3ccccc3)n2)cc1 ; Cc1ccccc1-c1csc(NC(=O)c2ccno2)n1]

In a further embodiment, the compound interacts with (i) domain 2 of gp130 and which locks gp130 into a non-permissive conformation, (ii) produce atypical gp130 homodimers and/or (iii) modulates STAT3 and/or MYC signaling.

In a particular embodiment, the disclosure further provides for engrafting chondrocytes made by a method disclosed herein to a subject in need thereof. In a further embodiment, the chondrocytes engrafted are derived from iPSCS made from the subject's cells.

### DESCRIPTION OF DRAWINGS

FIG. 1A-B provides for Scale up and formulation of CGMP-grade hES-derived chondrocytes. (A) Exemplary schematic/flow process production of chondrocytes in 2 different formulations from ESI-017 cells. Pre-chondrocytes were seeded onto clinically-used porcine collagen I/III membranes (M) or aggregated to generate chondrospheres (CS). Cells were expanded and then cryopreserved under optimal conditions described in FIG. 7. (B) qPCR for chondrogenic and pluripotent genes at different stages of in vitro differentiation or in vivo fetal ontogeny (14-17 weeks). n=3-4 different batches or biological replicates (fetal); data presented as box and whisker plots showing all points.

FIG. 2A-Mprovides for transcriptional profiling of membrane embedded hESDC-M. (A) t-SNE plot of 1173 single cells sequenced, generated from ESI-017 cells (n=1, top) or hES-derived chondrocytes digested from membranes at d40 of differentiation (n=2 batches, bottom left and right, respectively). (B, C) t-SNE plots depicting expression of indicated genes at single cell resolution. (D) Violin plots for gene expression of selected chondrogenic genes; fetal chondrocyte expression data are shown for reference. (E) Selected gene ontology (GO) categories enriched in hESDC-M vs. ESI-017 cells based on genes with FDR<0.05,>2-fold change. (F) Re-clustering, (G) k-means clustering and (H) PRG4 and COL2A1 expression levels of 965 hESDC-M cells. (I) Venn diagrams demonstrating overlap of genes strongly enriched (biomarker genes) in the indicated cluster with (top) genes enriched in embryonic chondroprogenitors isolated from 5-6 wk limbs vs. fetal chondrocytes isolated at 17 wks from knee joints analyzed by scRNA-Seq or (bottom) vice versa. (J-M) Expression of selected and biomarker genes in each cluster of hESDC-M.

FIG. 3A-C shows focal articular cartilage defects treated with hESDC-M have improved repair at 6 months. (A) Gross visual appearance of all 10 defects created in the femoral condyle of control (membrane alone, top row) or treated (hESDC-M, bottom row) Yucatan minipig knees after 6 months. Scale bar=10 mm. (B) Safranin O/Fast Green staining of the interface between the graft and endogenous tissue or the defect itself (boxes); where the boxed regions are shown at higher magnification below. Scale bar=100 μm. (C) Histological scoring of sections from control and treated femoral condyles for the 14 parameters comprising the ICRS II cartilage repair scoring system (left); each point represents the average of both defects per animal. (Right) Aggregate score of all 14 parameters over the 10 defects scored. Identifiers above or under images represent each animal. p-value was calculated using unpaired Student's t-test; data presented as mean±SD.

FIG. 4A-C provides hESDC-M treated defects evidence superior repair and contain both human and pig cells at 6 months. (A) Histochemical staining of the full defect (indicated by arrows) for Safranin O/Fast Green to assess glycosaminoglycans for control (membrane only) and treated (hESDC-M) animals. Representative images of immunohistochemical staining of the boxed area for human-specific antigen Ku80 and zonal markers of articular cartilage for both control and treated femoral condyles are shown and highlighted with black triangles; scale bar=200 μm. (B) Quantification of Ku80 +cells (mean±SD of 5 biological replicates). (C) qPCR analysis of human TERT gene. Standard curve constructed with human chondrocyte genomic DNA allowed reliable detection of as few as 100 human cells (mean±SD of 3 biological replicates). d Genomic DNA extracted from the indicated tissues was analyzed for the human TERT gene. Representative amplification plots are shown; human cells were detected in all defects of animals treated with hESDC-M. PBMCs=peripheral blood mononuclear cells.

FIG. 5A-C demonstrates that hESDC-M elicit biomechanically superior articular cartilage repair long term in porcine knees at 6 months. (A) Analysis of biomechanical properties was carried out using Mach-1 scanning indenter (Biomomentum, Canada). At least 10 points were analyzed within each defect. Heat maps representing instantaneous modulus and thickness are shown for each group. Mapping is artificial to illustrate the differences between specimens; scales for each measurement are shown. (B) Quantitative assessment of instantaneous modulus within the healing defect area 1 month after transplantation of 2 doses of either chondrospheres (CS) or collagen I/III membrane embedded hES-derived chondrocytes (CMC). The best repair was observed in the high dose CMC group. Data presented as mean±SD of aggregate values for 4 defects per each group; p values were calculated via one-way Anova followed by Tukeys test.

FIG. 6A-G shows hESDC-M produce paracrine factors that drive chondrogenesis of endogenous cells. (A) Schematic depicting the methylcellulose (MC) culture method created with Biorender.com. (B) Clonogenicity of porcine bone-marrow derived stromal cells (pBMSCs) in MC with different GFs; n=4 biological replicates. Representative image of a pBMSC-derived colony after 4 weeks in MC with 3 growth factors (right); scale bar=100 μm. (C) Alcian Blue and Toluidine Blue staining (left, middle) and immunohistochemical staining various chondrogenic markers of pBMSCs grown in MC with 3 GFs after 4 weeks. Scale bar=100 μm. (D) qPCR of chondrogenic genes (n=5 biological replicates for P1 pBMSCs and PO Ch, n=4 for pBMSCs in MC, and n=3 for pBMSCs cultured micromass). (E) Schematic of the MC with Transwell culture method. (F) Clonogenicity of pBMSCs in MC with a membrane only or hESDC-M in Transwell after 4 weeks, n=3 biological replicates per group. Representative images of pBMSCs in the Transwell after 4 weeks are shown; scale bar=100 μm. (G) qPCR of chondrogenic genes from pBMSCs grown in Transwell with hESDC-M, n=4 biological replicates. p-values were calculated with an unpaired Student's t-test; data presented as mean±SD or box and whisker plots showing all points.

FIG. 7 shows optimization of cell cryopreservation. Cell viability was assessed using Live/Dead assay prior to viably freezing and within 4 hours after thawing. Mesencult™ ACF provided the best viability post-thaw and was used for all subsequent preparations. p values were calculated via one-way ANOVA followed by Tukey's test; data are presented as mean±SD of 3 experimental replicates.

FIG. 8A-C provides an overview of surgical procedure and assessment of short-term cartilage repair at 1 month. (A) Assessment of proteoglycan content via Safranin O staining of healthy cartilage and groups one month after implantation; images show the junction of intact articular cartilage with the defect area, with boxes denoting the regions shown at higher magnification. (B) ICRS II aggregate scoring of defects calculated via 14 parameters of the ICRS II Cartilage Repair Scoring System demonstrated the high dose, membrane embedded formulation of hES-derived chondrocytes provided superior short-term repair. (C) Representative images of Ku80+ human cells in the membrane only or hESDC-M after 1 month. n=4 defects per condition, 2 defects per knee. Scale bars=100 μm; data presented as mean±SD. p values were calculated via one-way ANOVA followed by Tukey's test.

FIG. 9 demonstrates quantitative assessment of instantaneous modulus within the healing defect area 1 month after transplantation of 2 doses of either hESCderived chondrospheres (hESDC-CS) or collagen I/III membrane embedded hESDC-M. The best repair was observed in the high dose hESDC-M group. Data presented as mean±SD of aggregate values for 4 defects per each group; p values were calculated via one-way ANOVA followed by Tukey's test.

FIG. 10A-E shows immunological response to hESDC-M. (A) Representative images of immunohistochemical staining for immune cell markers in the cartilage defect at 1 month post implantation of the membrane only (left column) or the hESDC-M (right column). (B) Representative images of immunohistochemical staining for immune cell markers in the cartilage defect at 6 months post implantation of the membrane only (left column) or the hESDC-M (right column). (C) Representative Hematoxylin and Eosin (H&E) stains and quantification of synovial characteristics 1 month after implantation using previously described methods29 (n=4 biological replicates for membrane only and hESDC-M group (D) Representative H&E stain and corresponding quantification of synovium 6 months after implantation; left image is the membrane only, right image is the defect with hESDC-M; (n=5 biological replicates for membrane only group, n=5 for hESDC-M group) (E) Representative Toluidine Blue stain of healthy articular cartilage and cartilage with the defect area 6 months after implantation; top image is the membrane only, bottom image is the defect with hESDC-M, and right is the healthy cartilage. All scale bars=100 μm, p values calculated with an unpaired t-test and data is presented as mean±SD.

FIG. 11A-D shows that hESDC-M are not contaminated by hESCs and represent more immature chondrogenic cells than hBMSC-M. (A) t-SNE and violin plots depicting expression of indicated genes at single cell resolution. (B) qPCR of superficial gene expression (n=7 independent batches; p values were calculated with an unpaired ttest; data presented as box and whisker plots. (C) Violin plots for gene expression of selected chondrogenic genes in hESCs, hESDC-M and human bone marrow stromal cells cultured on membranes (hBMSCs-M). (D) Violin plots for gene expression of selected stromal genes.

FIG. 12A-F demonstrates chondrogenic ontogeny at the single cell level. (A) t-SNE plot of single cell sequencing data generated at 4 stages of human chondrocyte ontogeny. (B) Cell trajectory analysis of Col2+ cells from human in vivo ontogeny and cultured hESDC-M and ESI-017 cells constructed using Monocle348. Violin plots for gene expression of selected (C) superficial, (D) transitional and (E) deep zone genes. (F) Gene sets were created by intersecting two data sets generated with bulk RNA-seq of embryonic chondroprogenitors and fetal chondrocytes. Genes enriched in “pre-chondrocytes” vs. “resting chondrocytes” were overlapped with genes enriched in “embryonic 5-6 WPC (weeks post conception)” and “17 WPC” and vice versa to create lists of common genes enriched in embryonic chondroprogenitors and fetal chondrocytes. Venn diagrams demonstrating overlap of biomarker genes strongly enriched and representative of the indicated cluster of hESDC-M (see FIG. 2h) analyzed by scRNA-Seq with gene lists generated by bulk RNA-Seq.

FIG. 13A-E shows levels of chondroinductive paracrine factors of hESDC-M and hBMSCs. qPCR for various (A) TGF-B, (B) FGF, and (C) BMP family members in P1 hBMSCs (n=3 biological replicates of 27-29 yo) and hESDC-M (n=7 batches). (D) ELISA analyses of representative growth factors (BMP-2, TGF-31, & FGF2) secreted by P1-3 hBMSCs (n=7 biological replicates aged 19-64 yo) and hESDC-M (n=9 batches for FGF₂, n=7 batches for TGF-31 and n=6 batches for BMP-2). p values calculated with unpaired t-test; error bars represented as mean±SD. (E) Violin plots depicting gene expression in ESI-017 cells and 2 replicates of hESDC-M analyzed by scRNA-Seq.

FIG. 14A-E shows endogenous articular chondrocytes maintain their chondrogenic profile in the presence of hESDC-M. (A) Representative image of two pig chondrocyte (Ch) colonies (scale bar=500 μm) & clonogenicity of pig chondrocytes (n=4 biological replicates) cultured in methylcellulose (MC) for 4 weeks. (B) qPCR of chondrogenic genes (n=4 biological replicates). (C) Representative image of a pig Ch colony in MC after 4 weeks of culture with hESDC-M in a Transwell. Clonogenicity of pig Ch in MC with either an empty membrane or hESDC-M in a Transwell after 4 weeks (n=4 biological replicates per group). Scale bar=100 μm. (D) qPCR of chondrogenic genes in pig Ch grown in Transwell culture with hESDC-M (n=4 biological replicates). (E) Alcian Blue and Toluidine Blue staining (left, middle left) and immunohistochemical staining of various chondrogenic markers of pig chondrocytes grown in MC with 3 GFs after 4 weeks. Scale bar=100 μm; all p values calculated with unpaired t-test; error bars represented as mean±SD.

FIG. 15 shows immunohistochemical characterization of healthy porcine cartilage. Representative images of immunohistochemical staining of healthy porcine articular cartilage for human-specific antigen Ku80 and cartilage zonal markers; scale bar=200 μm.

### DETAILED DESCRIPTION

As used herein and in the appended claims, the singular forms “a,” “an, ” and “the” include plural referents unless the context clearly dictates otherwise. Thus, for example, reference to “a growth factor” includes a plurality of such growth factors and reference to “the stem cell” includes reference to one or more stem cells and equivalents thereof known to those skilled in the art, and so forth.

Also, the use of “or” means “and/or” unless stated otherwise. Similarly, “comprise,” “comprises,” “comprising” “include,” “includes, ” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this invention is not limited to the particular methodology, protocols, and reagents, etc., described herein and as such may vary. The terminology used herein is for the purpose of describing particular embodiments only and is not intended to limit the scope of the present invention, which is defined solely by the claims.

Any specific values should be understood to mean that they are within reasonable error for time, temperature, range, etc. For example, reference to “2 minutes” should be construed as being preceded by “about”, wherein the value has reasonable error for processing and the like. In another embodiment, the term “about” includes a range of 1%-5%±the defined value.

Other than in the operating examples, or where otherwise indicated, all numbers expressing quantities of ingredients or reaction conditions used herein should be understood as modified in all instances by the term “about.” The term “about” when used to described the present invention, in connection with percentages means ±1%-5%.

As used herein, the term “collagen, ” can mean, but is in no way limited to, any of a family of extracellular, closely related proteins occurring as a major component of connective tissue, giving it strength and flexibility. At least 14 types exist, each composed of tropocollagen units that share a common triple-helical shape but that vary somewhat in composition between types, with the types being localized to different tissues, stages, or functions. In some types, including the most common, Type I, the tropocollagen rods associate to form fibrils or fibers; in other types the rods are not fibrillar, but are associated with fibrillar collagens, while in others they form nonfibrillar, nonperiodic, but structured networks. Cartilage can contain chondrocytes or chondrocyte-like cells and intracellular material, proteoglycans, and other proteins. Cartilage includes articular and non-articular cartilage.

“Articular cartilage, ” also referred to as hyaline cartilage, refers to an avascular, non-mineralized connective tissue, which covers the articulating surfaces of bones in joints and serves as a friction reducing interface between two opposing bone surfaces. Articular cartilage allows movement in joints without direct bone-to-bone contact. The cartilage surface appears smooth and pearly macroscopically, and is finely granular under high power magnification. Articular cartilage is associated with the presence of Type II and Type IX collagen and various well-characterized proteoglycans, and with the absence of Type X collagen, which is associated with endochondral bone formation.

The term “cell” can mean, but is in no way limited to, its usual biological sense, and does not refer to an entire multicellular organism. The cell can, for example, be in vivo, in vitro or ex vivo, e.g., in cell culture, or present in a multicellular organism.

As used herein, the term “stem cells, ” can mean, but is in no way limited to, undifferentiated cells having high proliferative potential with the ability to self-renew that may generate daughter cells that may undergo terminal differentiation into more than one distinct cell phenotype. These cells may be able to differentiate into various cells types and thus promote the regeneration or repair of a diseased or damaged tissue of interest in vivo, in vitro or ex vivo. The term “cellular differentiation” refers to the process by which cells acquire a cell type. The term “progenitor cell” as used herein refers to an isolatable cell of any lineage that maintains the plasticity to differentiate into one or more target cell type that includes, but is not limited to, chondrocytes, osteocytes, and adipocytes. A progenitor cell, like a stem cell, may be able to differentiate into a specific type of cell, but is already more specific than a stem cell, and is pushed, or stimulated, to differentiate into its “target” cell type. Generally, stem cells can replicate indefinitely, whereas progenitor cells can only divide a limited number of times.

As used herein, the terms “osteoprogenitor cells”, “chondroprogenitor cells”, “osteochondroprogenitor cells”, “mesenchymal cells”, “mesenchymal stem cells (MSC)”, or “marrow stromal cells” are used interchangeably to refer to multipotent stem cells that can differentiate along one or several lineage pathways into osteoblasts, chondrocytes, myocytes, adipocytes, and tendocytes.

As used herein, the term “chondrocytes” can mean, but is in no way limited to, cells found in cartilage that produce and maintain the cartilaginous matrix. The term “chondrogenesis” refers to the formation of new cartilage from cartilage forming or chondrocompetent cells.

The terms “patient”, “subject” and “individual” are used interchangeably herein, and refer to an animal, particularly a human, to whom treatment including prophylaxis treatment is provided. This includes human and non-human animals. The term “non-human animals” and “non-human mammals” are used interchangeably herein includes all vertebrates, e.g., mammals, such as non-human primates, (particularly higher primates), sheep, dog, rodent (e.g., mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. “Mammal” refers to any animal classified as a mammal, including humans, non-human primates, domestic and farm animals, and zoo, sports, or pet animals, such as dogs, cats, cattle, horses, sheep, pigs, goats, rabbits, etc. Patient or subject includes any subset of the foregoing, e.g., all of the above, but excluding one or more groups or species such as humans, primates or rodents. A subject can be male or female. A subject can be a fully developed subject (e.g., an adult) or a subject undergoing the developmental process (e.g., a child, infant or fetus).

The term “treat” or “treatment” as used herein, refers to a therapeutic treatment wherein the object is to eliminate or lessen a condition or a symptom. Beneficial or desired clinical results include, but are not limited to, elimination of symptoms or a condition, alleviation of symptoms or a condition, diminishment of extent of the condition or symptom, stabilization (i.e., not worsening) of the state of the condition or symptom, and delay or slowing of progression of the condition or symptoms.

Cell therapy has been used successfully in the clinic for more than 50 years in the form of hematopoietic stem cell transplantation₁. Cell therapy, however, illuminated the need for HLA-matched donors due to graft versus host disease (GVHD) encountered during allogenic transplants, in which donor lymphocytes reacted against host tissues. In the case of allogenic solid organ transplantation, immunosuppression of the host is often required for extended periods. These aforementioned limitations in the availability and compatibility of donor tissue have prompted the search for other solutions which now potentially include embryonic stem cell (ESC) and induced pluripotent stem cell-derived (iPSC) cells and tissues. The field of pluripotent stem cell (PSC)-based regenerative medicine has advanced quickly as both iPSC- and ESC-derived cells are in clinical trials₅, with transplants into immunoprivileged sites such as the eye leading the way.

In the orthopedic field, reparative therapy for articular cartilage defects has classically relied on endogenous cells via the microfracture technique. In this procedure, channels are created into the bone marrow to allow mesenchymal cells with chondrogenic potential to enter the defect and generate neocartilage. More recently, autologous chondrocyte implantation (ACI) and variations thereof including MACI (matrix-associated ACI) that rely on expansion and reimplantation of chondrocytes from the patient, have been adopted. The long-term results of ACI-based procedures appear superior to microfracture, with reduced graft failure and improved patient-reported outcomes. Despite overall satisfactory outcomes, many patients still experience complications including graft integration failure, inferior quality of neocartilage (hyaline vs. fibrocartilage), donor site morbidity and osteoarthritis. Additionally, these approaches require two surgical procedures to perform the actual repair. This has fueled the search for allogeneic sources that may provide more cells with superior chondrogenic capacity without immunocompatibility issues or the need for multiple surgeries including juvenile cartilage and PSCs.

Generation of articular chondrocytes from PSCs has been challenging as most chondrogenic cells during development are fated to undergo hypertrophy and endochondral ossification rather than adopt an articular chondrocyte identity. Provided herein is the generation articular-like chondrocytes from GFP⁺ human pluripotent stem that can engraft, integrate into and repair osteochondral defects in subjects. Moreover, these human cells produce all layers of hyaline cartilage after 4 weeks in vivo, including a PRG4³⁰ superficial zone.

Further provided herein is methods that allow for the large-scale production of said articular-like chondrocytes. The methods disclosed herein allowed for the assessment of long-term, clinically relevant functionality of the articular-like chondrocytes. In particular, assessment was performed with Yucatan minipigs. Yucatan minipigs provide an excellent model for pre-clinical assessment of potential orthopedic therapies due to structural similarities, comparable thickness of articular cartilage and the ability to create defects of substantial volume; in addition, their size allows for cost-efficient care and observation for extended periods of time. As shown in the studies presented herein, long-term functional repair of porcine full-thickness articular cartilage defects could be repaired with hyaline cartilage from clinical grade hESC-derived articular chondrocytes that were produced using the large-scale production techniques and methods of the disclosure.

In particular, the studies presented herein demonstrate that hES-derived chondrocytes administered as a cryopreservable, membrane-embedded formulation, supported clinically relevant, long-term repair of full-thickness articular cartilage defects in pigs. At the molecular level, the membranes were found to contain no detectable residual hES cells and be populated with chondrocytes resembling both fetal and juvenile articular chondrocytes. Based on the high expression levels of SOX5/6/9 and genes associated with proliferation, it is likely that these immature articular chondrocytes mature in vivo to upregulate matrix production and assume a proper zonal identity. Importantly, the hyaline cartilage found in repaired defects had similar biomechanical properties to naive tissue, further supporting the concept that hES-derived chondrocytes can adopt adult-like properties upon transplantation and integration with surrounding cells and/or support recruitment and differentiation of chondrogenic cells into hyaline cartilage.

Further, the studies presented herein demonstrate that the hES-derived chondrocytes made by the processes disclosed herein did not induce local inflammation or immune cell infiltration despite being a xenograft in immunocompetent recipients with no immunosuppression. The results are in direct contrast to other studies that have demonstrated that xenografted articular chondrocytes in the knee can elicit a severe immune response. Thus, the processes disclosed herein represent a definite improvement when compare with similar techniques. The significant biomechanical improvement seen in porcine cartilage defects treated with human cells is very likely the result of both autocrine and paracrine mechanisms. This suggests that factors secreted by hES-derived chondrocytes can recruit endogenous cells capable of producing hyaline cartilage and support their differentiation down this path. Several candidate factors have been identified that could act in concert to achieve this result, including IGF-1, and TIMP-1 and -2.

IGF-1 is a classic anabolic factor for chondrocytes, promoting proteoglycan synthesis and reducing catabolismthis factor can also promote chondrogenesis from mesenchymal stem cells as has also been shown for FGF-6.

TIMP-1 and -2 are inhibitors of matrix metalloproteinases and have shown to be chondroprotective and prevent matrix loss downstream of the pro-inflammatory cytokine IL-1β. Following the exposure of the knee joint and creation of the defects, significant local inflammation occurs, driving production of catabolic enzymes and promoting fibrocartilage formation which may be ameliorated via the cell-mediated delivery of counteracting proteins such as TIMP-½ and IL-1RA (a decoy receptor for the pro-inflammatory cytokine IL-1α43). Together, these potential chondrogenic paracrine factors may modulate the microenvironment of the defect to promote migration and differentiation of endogenous cells into articular cartilage.

In one embodiment, the following methodology is used:

### Reagents

| | MANUFACTURER/ | |
|---|---|---|
| ITEM | DISTRIBUTOR | PART # |
| Y27632 | Tocris | 1254-10mg |
| mTeSR 1 (or mTeSr Plus) | Stemcell | 85850 |
| | Technologies | (or 100-0276) |
| ReLeSr | Stemcell | 100-0484 |
| | Technologies | |
| Human/Murine/Rat | Peprotech | 120-14E |
| Activin-A 10 ug | | |
| Human BMP4 50 ug | Peprotech | 120-05ET |
| Human b-FGF (FGF2) 50 ug | Peprotech | AF-100-18B |
| Human IGF-1 10 ug | Peprotech | 100-11 |
| Human LIF 50 ug | Peprotech | AF-300-05 |
| Human Noggin 50 ug | Peprotech | 120-10c |
| Human Sonic Hedgehog | Peprotech | 100-45 |
| (SHH) 25 ug | | |
| Human TGFb-1 10 ug | Peprotech | AF-100-21C |
| Human Wnt3a 10 ug | R&D Systems | 5036-WN |
| 1X PBS w/o Ca⁺⁺ Mg⁺⁺ | Corning | 21-031-CM |
| 35% BSA | Sigma | A7979 |
| Cell culture grade water | Cytivia | SH30529.02 |
| 12M (37%) Hydrochloric | Sigma | 320331 |
| Acid (HCl) | | |
| 1M Tris | Growcells.com | MRGF-4220 |
| Trehalose | Acros Organic | 182550250 |
| X-Vivo ™15 | Lonza | 04-418Q |
| TrypLe Select Enzyme | Thermo | 12563029 |
| (1x), no phenol red | Scientific | |
| CD326-Microbead | Miltenyi | 130-061-101 |
| CD309-Biotin (KDR) | Miltenyi | 130-093-603 |
| Streptavidin | Miltenyi | 130-048-102 |
| EDTA | Affymetrix | 15701 |
| MesenCult ACF Freezing | Stemcell Tech | 05490 |
| Media | | |

Exemplary procedure of 3D Culture: Pre-treat culture flask/plat with Y27632 (final concentration is 10 uM) for 2 hours. Aspirate mTeSR media from 2D culture flask comprising stem cells. Wash cells with appropriate volume of DPBS without Ca++/Mg++. Add 20 mL of ReLeSR and swirl the flasks to ensure entire cell surfaces are exposed to ReLeSR. Incubate for about 1 min at room temperature. Aspirate ReLeSR and leave thin layer of liquid to prevent drying of the flask/plate. Incubate flask/plate in the incubator for about 2-7 minutes. Add an appropriate volume of mTeSR to the plate/flask. Detach colonies (e.g., approximately 0.5 mm colonies, and colonies should not be disaggregated). Transfer detached cells to a spinner flask. Transfer the spinner flask to a slow speed stirrer plate in the incubator (up to 60 RPM). Culture it up to 7 days—change media every day.

Exemplary procedure of Cell Differentiation and purification: MI-A media—Add X-Vivo media to a filtration unit with 0.2 μm filter without connecting to a vacuum line. Add 10 mM Y27632 (Final conc 10 uM), 10 ug/mL Wnt3a (final conc 10 ng/ml), 10 ug/mL bFGF (final conc 10 ng/ml), and Activin A (final conc 10 ng/ml). Filter the solution. Take out a spin flask from the slow-speed stirrer for minimum of about 30 min (up to about 1 hour). Remove about 50%-70% of the mTeSR media. Transfer the rest of media to conical spin flasks while maintaining cell aggregation. Add an appropriate volume of MI-A media to the spin flask to avoid dry out. Centrifuge the conical tube for about 3 minutes at about 300 g. Aspirate supernatant. Resuspend with an appropriate volume of MI-A media and transfer to the spin flask. Wash conical tubes with appropriate volume of MI-A media and transfer to the spin flask. Transfer the spin flask to the slow speed stirrer in the incubator. Set spin speed about 45 rpm. Incubate on the slow speed stirrer in the incubator for about 3 days.

Prepare MI-B media by adding X-Vivo media to a filtration unit with 0.2 um filter without connecting to a vacuum line. Add Wnt3a (final conc 10 ng/ml), bFGF (final conc 10 ng/ml), and Noggin (final conc 50 ng/ml). Connect the vacuum line and filter it through filtration unit. Take out a spin flask from the slow-speed stirrer for minimum of about 30 min (up to about 1 hour). Remove about 50%-70% of the MI-A media. Transfer rest of media to conical spin flasks and try not to disrupt cell aggregation. Add an appropriate volume of MI-B media to the spin flask to avoid dry out. Centrifuge the conical tube for about 3 minutes at about 300 g. Aspirate supernatant. Resuspend with appropriate volume of MI-B media and transfer to the spin flask. Wash conical tubes with appropriate volume of MI-B media and transfer to the spin flask. Transfer the spin flask to the slow speed stirrer in the incubator. Set spin speed about 45 rpm. Incubate on the slow speed stirrer in the incubator for about 3 days.

Inducing chondrogenesis: Prepare CI-A media by adding X-Vivo media to a filtration unit with 0.2 um filter without connecting to a vacuum line. Add bFGF (final conc 10 ng/ml), and BMP4 (final conc 10 ng/ml). Connect the vacuum line and filter it through filtration unit. Close cap tightly and warm it up inside of clean water bath. Remove a spin flask from the slow-speed stirrer for a minimum of about 30 min (up to about 1 hour). Remove about 50%-70% of the MI media. Transfer the rest of media to conical spin flasks while not disrupting cell aggregation. Add an appropriate volume (depending upon tissue culture size) of CI-A media to the spin flask to avoid dry out. Centrifuge the conical tube for about 3 minutes at about 300 g. Aspirate supernatant. Try to avoid touching pellets. Resuspend with appropriate volume of CI-A media and transfer to the spin flask. Wash conical tubes with appropriate volume CI-A media and transfer to the spin flask. Transfer the spin flask to the slow speed stirrer in the incubator. Set spin speed about 45 rpm. Incubate on the slow speed stirrer in the incubator for about 3 days.

Prepare CI-B media: Add a X-Vivo media to a filtration unit with 0.2 um filter without connecting to a vacuum line. Add Y27632 (Final conc 10 uM), IGF-1 (final conc 10 ng/ml), bFGF (final conc 10 ng/ml), BMP4 (final conc 50 ng/ml), and SHH (final conc 25 ng/ml). Filter it through filtration unit.

Vitronectin Coating of tissue culture substrate (following a product manual from Stemcell Technologies). Thaw Vitronectin XF (Stemcell Tech; Cat#07180) and bring CellAdhere Dilution Buffer (Stemcell Tech; Cat#07183) at room temperature. Dilute Virtonectin XF in CellAdhere Dilution Buffer to reach a final concentration of 10 ug/mL. Gently mix Vitronectin XF (do not vortex). Use the diluted Vitronectin XF to coat tissue culture plate. Gently rock the tissue culture substrate back and forth to spread the Vitronectin XFM solution evenly across the surface. Incubate at room temperature for at least 1 hour before use. Do not let the Vitronectin XFM solution evaporate. Gently tilt the tissue culture substrate on to one side and allow the excess Vitronectin XF™ solution to collect. Remove the excess solution using a serological pipette or by aspiration. Ensure that the coated surface is not scratched. Wash the tissue culture substrate once using CellAdhere™ Dilution Buffer (e.g. 1 mL/well if using a 6-well plate). Aspirate wash solution and add the appropriate volume of culture medium (e.g. 2 mL/well if using a 6-well plate).

Take out a spin flask from the slow-speed stirrer for minimum 30 min (up to 1 hour). Pre-warm 1× Tryple select media. Take out the spin flask from the incubator and aspirate 50% of excessive media. Add 50 uL of 10 mM Y27632 to the spin flask. Put spin flask to the slow speed stirrer in the incubator and incubate for about 1 to 2 hours before magnetic sorting if that will take place (MACS). Take out a spin flask from the slow speed stirrer for a minimum of about 30 min (up to about 1 hour). Take out the spin flask from the incubator and aspirate excessive media (try not to aspirate aggregates). Transfer all media to a conical tube. Wash the spin flask with cold PBS and transfer to conical tube. Centrifuge for about 3 minutes at about 300 g. Aspirate supernatant (try not to touch a pellet); If cell pellet is too big, split it to couple of tubes. Wash the pellet with cold PBS and centrifuge for about 3 minutes at about 300 g. Aspirate supernatant in the conical tubes. Resuspend the pellet with an appropriate volume of pre-warmed 1× Tryple select. Incubate the conical tube in the incubator (37° C.) for about 2 minutes. Take out the tube and pipette up and down several times. Incubate the tube in the incubator for another approximate 2 minutes. Take out the tube and pipette up and down to disrupt cell aggregates to single cells. Wash with cold PBS. Centrifuge conical tube for about 3 minutes at 300 g. Carefully aspirate supernatant. Resuspend cell pellet with CI-B media. Put flask in the incubator for 24 hours. Exemplary cell density and media volumes:

| Plate | Cell density | Volume of CI-B media | |
|---|---|---|---|
| 24 well plate | 100,000/well | 0.5-1 | mL |
| 6 well plate | 900,000-1*10⁶/well | 1-3 | mL |
| T-25 cm2 flask | 2.5*10⁶/flasks | 3-5 | mL |
| T-75 cm2 flask | 7.5* 10⁶/flasks | 8-15 | mL |
| T-225 cm2 flask | 22.5*10⁶/flasks | 30-50 | mL |

Prepare collagen membrane: Take out collagen membrane. Transfer collagen membrane to sterile tissue culture substrate. Rough side should be facing up and smooth side should be facing bottom of tissue culture substrate. Pre-soak collagen membrane with CI-B media. Incubate at 37 degrees.

Seeding cells on collagen membrane: Take out tissue culture from the incubator. Removed CI-B media and wash with DPBS without Ca++and Mg++. Remove DPBS and add an appropriate volume for the tissue culture substrate of ReleSR or 1× Tryple Select. Make sure all cell surfaces are exposed to ReleSR for a minute. Remove ReleSR and incubate in 37 degrees for about 2-3 minutes. Add an appropriate volume of CI-B media to neutralize. Transfer all cells to conical tube. Count cell number using an automated hematocytometer with Trypan blue. Seed cells on the collagen membrane (5 million cells per cm²) in a dish/plate. Incubate dish/plate in hypoxia chamber for 3-4 days.

Maintenance: Prepare Maintenance media (if collagen membranes cultures in bioreactor, scale up the media proportionally): Add a X-Vivo media to a filtration unit with 0.2 um filter without connecting to a vacuum line. Add IGF-1 (final conc 10 ng/mL), bFGF (final conc 10 ng/ml), LIF (Final conc 50 ng/ml), TGFb1 (final conc 10 ng/ml), and BMP4 (final conc 1 ng/ml). Connect with the vacuum line and filter it through filtration unit. Remove dish/plate containing collagen membrane seeded with cells from the hypoxia chamber. Remove CI-B media from dish/plate (try not to touch collagen membrane and keep wet)—Change media every 2-3 days. Incubate in hypoxia chamber up to day 40 starting from Mesodermal induction (MI-A).

Cryopreserve membrane: Add appropriate volume for vial size of MesenCult ACF freezing media with 5 uL of 10 mM Y27632 in a cryo vial. Take out a plate from the hypoxia chamber Using a tweezer and carefully transfer collagen membranes to cryo vials (e.g., carefully roll them up and put them in a cryo vial). Store cryo vials in freezing container and transfer freezing container to −80 for 24 hrs. Then transfer cryo vials to liquid nitrogen tanks for longer storage.

Collagen with seeded cells can be used (or thawed and used) for cartilage repair. The method includes implanting a collagen membrane composition of the disclosure containing seeded chondrocytes to a cartilage defect site. The collagen membrane can be sized or configured to fit the defect site. The collagen membrane can be sutured or glued in place.

The following examples are intended to illustrate but not limit the disclosure. While they are typical of those that might be used, other procedures known to those skilled in the art may alternatively be used.

### EXAMPLES

General Methods: For all experiments, independent experiments with biological replicates were employed to generate data. For in vitro experiments expected to yield large differences, standard practice of using 3 replicates was followed. All statistical methods are described in the figure legends.

Culture of pluripotent stem cells and generation of skeletal progenitors: For all experiments, research grade ESI-017 were used; the stock line was purchased from BioTime. Cells were cultured on ESC-grade Matrigel (Corning) in mTesR1 media and passaged using ReLeSR (Stemcell Technologies) according to manufacturer's instructions. Batch scale production of ESI-017 cells was carried out in CellSTACK-5 flasks, yielding ˜1 billion cells per run. Each batch was tested for mycoplasma contamination by PCR (Sigma). Cells were then transferred to suspension culture in mTesR¹ for up to 5 days in spinner flasks (60 RPM). Differentiation was conducted as described in Ferguson et al. (Nature Communications 9:3634 (2018)) and Evseenko et al. (Proceedings of the National Academy of Sciences 107: 13742 (2010)). Briefly, mTesR¹ was replaced with Mesoderm Induction Media A (MIM-A) for 3 days (X-Vivo containing ROCK inhibitor, FGF2, Wnt3a and Activin A) followed by MIM-B for 3 days (X-Vivo containing Wnt3a, Noggin and FGF-2). Media was then changed to Chondrogenic Induction Media A (CIM-A; X-Vivo containing BMP-4 and FGF-2) for 4 additional days. Skeletal progenitors were then isolated using MACS by depleting for CD326 and CD309; for 2 hours before starting MACS isolation, ROCK inhibitor was added to the cell culture media. The purity of cells isolated using MACS was routinely assessed using flow cytometry.

Generation of chondrospheres: Skeletal progenitors from MACS were then cultured in EZSphere plates (Nacalai USA) at 100,000 cells per well in CIM-B15 (X-Vivo containing Shh, ROCK inhibitor, BMP-4, FGF-2, IGF-1 and Primocin) in 5% oxygen for 3-5 days to form chondrogenic aggregates. After firm aggregates were verified with microscopy, they were transferred to a perfusion bioreactor (Applikon Biotech) in Maturation Media (MM; X-Vivo media containing FGF-2, BMP-4, Shh, IGF-1, LIF, TGF-31 and Primocin) until d40 of differentiation. Conditions in the bioreactor were kept at 37° C., 5% O₂, 5% CO₂ and 30 RPM. Fresh media was added at a rate of 1 mL/hour. Upon maturation, chondrospheres contained 5×10⁴ cells on average. At d40, chondrospheres were cryopreserved in MesenCult-ACF plus ROCK inhibitor (Y27623, 10 μM; Tocris) and stored in liquid nitrogen until use.

Generation of hES-derived chondrocytes on membranes: Skeletal progenitors were isolated via MACS and seeded onto porcine collagen I/III membranes (Cartimaix; Matricel) sized with a 6 mm biopsy punch at two different densities designed to yield 6×10⁵ or 3×10⁶ million cells. Membranes were then cultured in 5% oxygen for 3-5 days in CIM-B and then transferred to the bioreactor as above for chondrospheres with the exception that rotation was not started until 3 days after transfer of membranes and was at 15 RPM. At d40, membranes were cryopreserved using MesenCult-ACF as above for chondrospheres.

Generation of hBMSCs on membranes (hBMSC-M). Human bone marrow stromal cells (n=3 donors aged 19, 68, and 87 years (all P1); pooled) were seeded onto porcine collagen I/III membranes (Cartimaix; Matricel) sized with a 6 mm biopsy punch at a total cell number of 3×10⁶. Membranes were then cultured in 5% oxygen for 3-5 days in CIM-B, then maintained with MM until d40. At d40, membranes were digested for single cells and scRNA-sequencing was performed.

Optimization of cryopreservation media: Live cell numbers per batch of chondrospheres or membranes were determined prior to freezing using the Live/Dead Cell Viability Assay (Biovision). Chondrospheres or membranes were then cryopreserved following the manufacturer's instructions for each product (Prime XV FreezIS, Irvine Scientific; MesenCult-ACF, CryoStor CS5 or 10, mFreSR; Stemcell Technologies), thawed and then viability from the same batch compared to the starting viability before freezing.

Quantitative Real-Time PCR: Power SYBR Green (Applied Biosystems) RT-PCR amplification and detection was performed using an Applied Biosystems Step One Plus Real-Time PCR machine. The comparative Ct method for relative quantification (2-ΔΔCt) was used to quantitate gene expression. TBP (TATA-box binding protein) was used for gene normalization and expressed relative to a calibrator (sample in each set with lowest expression). Primer sequences used for qPCR are available on request. For quantification of human cell numbers in pig samples, a human TERT Taqman assay was used (Thermo). A standard curve was created with known numbers of human cells, which both determined the detection threshold as well as allowed calculation of human cell numbers in a sample based on Ct values.

Large animal model of articular cartilage repair. Yucatan minipigs were purchased from S & S farms at 6 months of age and housed under the supervision of the USC Department of Animal Resources (DAR). All preoperative, surgical and post-operative procedures were conducted following USC DAR guidelines and were overseen by the USC Institutional Animal Care and Use Committee (IACUC). Five animals per group (main study) with 2 defects each were included based on power calculations for significance. Calculation of animal numbers was based on the following statistical formula: n=1+2C(s/d)², where C is a constant, s is expected standard deviation and d is expected difference between means. Every care was been taken to minimize the number of pigs required. Power analyses were performed for all animal studies as follows: For all treatment groups, with an alpha of 5%, and power level of 80%, N=5 per treatment group is used. Estimated standard deviation and differences between means used for calculations are based on pilot studies. Animals were anesthetized for surgery using Telazol/Xylazine 2.2-4.4 mg/kg administered intramuscularly. Medial para-patellar arthrotomy was performed by inserting microsurgical scalpel medially and proximally to the insertion of the patellar tendon on the tibia and extending it proximally until the attachment of the quadriceps muscle. The medial margin of the quadriceps was separated from the muscles of the medial compartment. The joint was extended and the patella dislocated laterally. The joint was then be fully flexed to expose the patellar groove. Two full-thickness injuries 6 mm in diameter were made using a disposable biopsy punch. No randomization was applied. For animals receiving chondrospheres in the pilot study, surgical fibrin glue (Ethicon) with or without chondrospheres was applied directly to the defect areas. For animals receiving membranes, pre-sized membranes were applied to the defect and secured with fibrin glue. Arthrotomies were then relieved, wounds sutured and animals monitored for recovery. Following 1 or 6 months, animals were sacrificed after receiving anesthesia as above using pentobarbitol (100 mg/kg).

Tissue collection and digestion: Adult human primary tissue samples were obtained from National Disease Research Interchange (NDRI). Consented, de-identified human fetal tissues were obtained from Novogenix Laboratories or the University of California Los Angeles (UCLA) Center for AIDS Research (CFAR) Gene and Cellular Therapy Core following institutional review board (IRB) approval. Articular chondrocytes from the 7-year old human juvenile subject were obtained from leftover tissues from surgical procedures approved by the UCLA institutional IRB, with patient consent and de-identification. Use of human tissues was IRB exempt by the UCLA Office of the Human Research Protection Program (IRB #15-000959). Human primary tissues or hES-derived chondrocytes on membranes were manually cut into small pieces and digested 4-16 hrs at 37° C. with mild agitation in digestion media consisting of DMEM (Corning) with 10% FBS (Sigma), 1 mg/mL dispase (Gibco), 1 mg/mL type 2 collagenase (Worthington), 10 μg/mL gentamycin (Teknova) and 100 μg/ml primocin (Invivogen).

Immunohistochemistry (IHC): Tissues were fixed in 10% formalin and sectioned at 5 μm. For DAB, sections were deparaffinized using standard procedures and antigen retrieval was performed by bringing samples to a boil in 1× citrate buffer pH 6.0 (Diagnostic Biosystems), and incubating at 60° C.for 30 minutes followed by 15 minutes cooling at room temperature. Endogenous peroxidase activity was quenched by treating samples with 3% H₂O₂ for 10 minutes at RT. Sections were then blocked in 2% normal horse serum for 20 minutes. Sections were then incubated with primary antibodies diluted in TBS with 1% BSA (Sigma) overnight at 4° C. Sections were washed 3 times with TBS 0.05% Tween 20 (Sigma) (TBST) before addition of HRP-conjugated secondary antibody for 30-minute incubation at RT. Sections were washed 3 times with TBST after secondary incubation and DAB substrate was then added until positive signal was observed. Sections were then immediately washed with tap water, counterstained in hematoxylin for 30 seconds and washed again with tap water before dehydration and mounting. For Hematoxylin and eosin staining, sections were deparaffinized, rinsed in tap water, and stained with Hematoxylin for 3 minutes. Sections were then washed in tap water and stained with Eosin for 2 minutes before a final wash in tap water. Safranin O/Fast Green staining was performed. To quantitate cartilage repair, the ICRS II scoring system was employed by two blinded observers. Toluidine Blue and Alcian Blue staining was performed on deparaffinized sections in accordance with standard laboratory techniques.

Biomechanical assessment of defect repair: Freshly harvested porcine cartilage tissues 6 months of age were affixed to the sample holder of the Mach-1 Mechanical Tester (Biomomentum) using instant glue (Loctite 4013) and immersed in DMEM/0.9% NaCl (1:1). The Mach-1 configuration uses a spherical indenter tool attached to a highly sensitive multiaxial load cell and automated fine motor controller, allowing for compression of the cartilage by 30% of its thickness, and live recording of resultant forces generated in all x-y-z planes. The indenter tool is then replaced with a needle which penetrates the cartilage at each point until forces comparable to underlying bone are detected, allowing for accurate thickness measurement. Altogether, the forces generated during indentation and thickness measurements are used to calculate the instantaneous modulus, which reflects the elasticity, stiffness, and resistance to compression of the tissue. Each condyle was manually mapped for testing using the Biomomentum mapping software. On average, between 40-50 points were tested on each affected condyle, with no less than 10 points directly in the defect areas, and usually about 1-3 mm apart from each other on the surface. Each control condyle was tested for an average of 15-20 points, which were spaced about 2-5 mm apart. Mapping coordinates were input into the software and indentation analysis to map instantaneous modulus was performed with the 1 mm spherical indentor tool with the following parameters: Z-contact velocity: 0.1000 mm/s; contact criteria: 0.1003 N; scanning grid: 0.2000 mm; indentation amplitude: 0.300 mm; indentation velocity: 0.300 mm/s; relaxation time: 5s. To map thickness, the indentor tool was replaced with a 26 G ¾ inch hypodermic needle and the mapping executed with the following needle penetration parameters: stage axis: position z; load cell axis: Fz; direction: positive; stage velocity: 0.2000 mm/s; contact criteria: 2.000 N; stage limit: 15 mm; stage repositioning: 2× load resolution; offset: 0. Heat maps were generated with the Mach-1 software provided by Biomomentum.

Single-cell sequencing using 10X Genomics: Single cell samples were prepared using Single Cell 3/Library & Gel Bead Kit v2 and Chip Kit (10X Genomics) according to the manufacturer's protocol. Briefly samples were FACS sorted using DAPI to select live cells followed by resuspension in 0.04% BSA-PBS. Nearly 1, 200 cells/μl were added to each well of the chip with a target cell recovery estimate of 8,000 cells. Thereafter Gel bead-in Emulsions (GEMs) were generated using GemCode Single-Cell Instrument. GEMs were reverse transcribed, droplets were broken and single stranded CDNA was isolated. cDNAs were cleaned up with DynaBeads and amplified. Finally, cDNAs were ligated with adapters, post-ligation products were amplified, cleaned up with SPRIselect. Purified libraries were submitted to UCLA Technology Center for Genomics & Bioinformatics for quality check and sequencing. The quality and concentration of the purified libraries were evaluated by High Sensitivity D5000 DNA chip (Agilent) and sequencing was performed on NextSeq500.

10× sequencing data analysis: Raw sequencing reads were processed using Partek Flow Analysis Software. Briefly, raw reads were checked for their quality, trimmed and reads with an average base quality score per position>30 were considered for alignment. Trimmed reads were aligned to the human genome version hg38-Gencode Genesrelease 30 using STAR −2.6.1 d with default parameters. Reads with alignment percentage>75% were de-duplicated based on their unique molecular identifiers (UMIs). Reads mapping to the same chromosomal location with duplicate UMIs were removed. Thereafter ‘Knee’ plot was constructed using the cumulative fraction of reads/UMIs for all barcodes. Barcodes below the cut-off defined by the location of the knee were assigned as true cell barcodes and quantified. Further noise filtration was done by removing cells having>3% mitochondrial counts and total read counts >24,000. Genes not expressed in any cell were also removed as an additional clean-up step. Cleaned up reads were normalized using counts per million (CPM) method followed by log transformation generating count atrices for each sample. Samples were batch corrected on the basis of expressed genes and mitochondrial reads percent. Count matrices were used to visualize and explore the samples in further details by generating tSNE plots generated using default parameters in Partek. K-means clustering was computed for identifying groups of cells with similar expression profile using Euclidean distance metric based on the most appropriate cluster count. A maximum of 1000 iterations were allowed and the top marker features for each cluster was determined.

Gene ontology enrichment analysis for the differentially expressed genes was performed using DAVID Gene Functional Classification Tool ([http://]david.abcc.ncifcrf.gov; version 6.8). Dot plots and Violin plots were generated in R (v4.0.3) using ggplot2 (v3.3.3) package. Two-way Venn diagrams were generated using BioVenn. Hypergeometric p values were calculated assuming 25,000 human genes.

Trajectory analysis: Cell trajectories were constructed using Monocle3 package in R according to Trapnell lab guidelines (https://] [github. com/cole-trapnell-lab/monocle3). Count matrices, cell metadata and gene metadata were used to create Monocle3 object. A subset of genes that exhibit high cell-to-cell variation in the dataset was determined by directly modeling the mean-variance relationship inherent in the data using Seurat. Preprocessing was done by calculating significant PCs (principal components) ensuring usage of enough PCs to capture most of the variation in gene expression across all the cells in the data set. Variable genes obtained from Seurat were used to pre-process the data. The dimensionality of the data was thereafter reduced using uniform manifold approximation and projection (UMAP). Cells were plotted onto the UMAP space for visualization of their distribution, identification of cell types and community detection to group cells into clusters. Next the principal graph was learned within each cluster, trajectory was constructed and the cells were ordered to measure their progress in pseudotime.

Methylcellulose culture method of porcine BMSCs and chondrocytes. To assess the clonality and capacity of pig BMSCs to undergo chondrogenesis in vitro, porcine bone marrow stromal cells (pBMSCs) or articular chondrocytes were isolated from the distal femoral epiphysis or articular surface of the condyles, respectively, of 3-4-month-old Yucatan minipigs (S & S Farms). Tissues were digested as described above. Methylcellulose-based media (StemCell) was resuspended with DMEM/F12 (Corning)+10% FBS+1% P/S/A and either 10 ng/ml FGF-2, 10 ng/ml BMP-2, 10 ng/ml TGFβ-1, or all three growth factors, to make a 1% methylcellulose-based media. Either P1 BMSCs or PO chondrocytes were seeded in 6-well ultra-low attachment plates (Corning) at a low density (300 cells/ml) and cultured for 3-4 weeks. 120-160 μL of liquid DMEM/F12 with respective growth factors was applied to the surface of the wells to ensure moisture and nutrients remained available to the cells biweekly. For the Transwell-methylcellulose co-culture, hES-derived chondrocytes on membranes were placed in a 1 μm-pore Transwell insert (Falcon) with DMEM/F12+10% FBS+1% P/S/A. P1 MSCs or P0 chondrocytes were seeded in the same media with methylcellulose, and cultured in 24-well ultra-low attachment plates (Corning) at a low density (300 cells/ml) for 3-4 weeks. 40-60 μL of media was added biweekly to the methylcellulose as maintenance. Clonogenicity was calculated by manual counting of clones larger than ˜40 μm in diameter or greater than 5 cell divisions. All images of clones in methylcellulose were taken on an Echo Revolve Inverted microscope.

ELISA detection of proteins secreted by hESDC-M. Either 1×10⁶ human bone marrow stromal cells (hBMSCs; n=7 different donors) or whole hES-derived chondrocytes on membranes (n=6-9 batches) were lysed with 500 μL of 2× Lysis buffer (Ray Biotech) supplemented with a phosphatase/protease inhibitor (Thermo-Fisher). Lysates were then centrifuged to remove cellular debris, and a Bicinchoninic acid (BCA) protein assay (Thermo-Fisher) was performed to quantify total lysed protein. ELISAs for FGF-2 (Ray Biotech), BMP-2 (R & D Systems), and TGF-β1 (R & D Systems) were performed according to the manufacturer's protocols.

Antibody List: Please see Table 1 for a list of antibodies and dilutions used.

TABLE 1
Antibodies used in this study

| | | Catalog | |
|---|---|---|---|
| Antibody | Vendor | Number | Dilution |
| CD326-PerCP-Cy5.5 | BD Biosciences | 347199 | 10 uL/10⁶ cells |
| CD309-PE | R&D Systems | FAB357P | 10 uL/10⁶ cells |
| Collagen II | Abcam | ab185430 | 1:100-1:250 (IHC) |
| PRG4 | Abcam | ab28484 | 1:250 (IHC) |
| SOX9 | Abcam | ab26414 | 1:200 (IHC) |
| Collagen X | Abcam | ab58632 | 1:250-1:1000 (IHC) |
| Collagen I | Abcam | ab34710 | 1:250 (IHC) |
| Ku80 | Abcam | ab79391 | 1:250 (IHC) |
| CD3 | Protein Tech | 17617-1-AP | 1:500 (IHC) |
| CD68 | Bioss | BS-1432R | 1:50 (IHC) |
| Myeloperoxidase | Invitrogen | PA5-16672 | 1:50 (IHC) |
| Anti-Mouse IgG | Vector | MP-7422 | Pre-diluted |
| ImmPRESS | | | |
| Anti-Rabbit IgG | Vector | MP-7401 | Pre-diluted |
| ImmPRESS | | | |
| Mouse IgG Isotype | abCAM | Ab170191 | 1:50-1:1000 (IHC) |
| Control | | | |
| Rabbit IgG Isotype | Invitrogen | 02-6102 | 1:50-1:1000 (IHC) |
| Control | | | |

Detailed protocol for directed differentiation of pluripotent stem cells into chondrocytes in fully defined animal free conditions.

2D Culture: Propagation ESI-017 for MCB generation:
ESI-017 GMP grade is obtained from BioTime Inc. Pre-coat T75 culture flask (Corning) with Recombinant human Laminin 521 (rhLaminin-521) (Thermo A29249) diluted in PBS. To prepare a working solution Thaw rhLaminin-521 slowly at 2° C.to 8° C. Coating concentration of Laminin 521 is 0.5 μg/cm². 8 mL of the coating solution is made to coat one T75 Flask. Coat for at least 1 hour.
Thaw a vial of ESI-017 cells (1 million cells) is thawed quickly in a 37° C.water bath for 1-2 min until small ice crystals remain in the vial. Transfer all contents of the vial to a conical tube, slowly dilute freezing media with cells with 10× volume of pre-warmed mTeSR¹ Plus media (StemCell Technologies—05825) drop by drop.
Centrifuge at 300×g for 3 min.
Resuspend cells with 15 mL of mTeSR Plus and plate in a T75 flask.
Culture until 70-80% confluent. Change medium once in 2 days.
When 70% confluent transfer into a T225 flask (Corning CLS431082). For passaging add 3 mL of ReLeSR (STEMCELL Tech 05873) following manufacturer's instructions and incubate for 1 min at room temperature.
Remove ReLeSR and incubate in an incubator (37° C.) for 2 min without addition of any medium.
Add 10 mL mTeSR and collect all cells from 2D flask.
Centrifuge at 300×g for 3 min.
Resuspend cells with 40 mL of mTeSR Plus and transfer it (˜30 MLN cells) to a T225 flask.
Culture until 70-80% confluent. Change medium once in 2 days.
Coat a Polystyrene CellSTACK®—5 Chamber with Vent Cap (Corning 3313) with Laminin 521 as discuss above.
Passage cells from the T225 flask using 9 mL of ReLeSR as describe above.
Collect cells (˜100 million).
Centrifuge at 300 g for 3 min.
Resuspend cells with 500 mL of mTeSR Plus and plate in a CellSTACK®—5 flask.
Culture until 70-80% confluent. Change medium once in 2 days. An 80% confluent CellSTACK® flask will contain 1 billion cells. Expanded cells is characterized for markers of pluripotency and karyotyped prior to the generation of master cell bank (MCB). Cells is cryopreserved in liquid nitrogen vapor phase using cryo vials (Corning CLS430661) in CryoStor® CS10 (Catalog #07955) 1 MLN cells per 1 mL of medium per vial. Differentiation is initiated in the same flasks by replacing maintenance medium to differentiation medium as described by Ferguson et al.

3D Culture: Prior to differentiation cells from the ESI-017 PSC MCB is expanded in 2D system to 1 billion cells as described above, and then transferred (as described above) into 3-D conditions/spin culture using Corning® 500 mL disposable spin flasks (Corning-3153).
Cells are cultured in 500 mL of fully defined mTESR Plus medium (STEMCELL Tech) supplemented with commercially available ROCK inhibitor Y-27632 (10 μM final).
Cells are cultured up to 5 days (60 rpm); replace 50% of medium daily.
After 5 days remove the flasks from the spinning platform. Let the aggregates settle for 15 minutes.
Aspirate mTESR Plus and replace it with differentiation medium as described below.
   Mesoderm Induction (MI)—A Day (3 days)
Rock Inhibitor (Y27632) 10 μM
Wnt3a 10 ng
Activin A 10 ng
FGF₂ 10 ng
X-Vivo (Lonza 04-743Q)
   MI-B Day (3 days)
WNT3a 10 ng
Noggin 50 ng
FGF₂ 10 ng
X-Vivo (Lonza 04-743Q)

### Chondrogenic Induction (CI-A)—Day (3 Days)

X vivo (Lonza 04-743Q)
FGF₂ 10 ng
BMP4 10ng

### MACS DAY—Magnetic Sorting:

Remove excessive media and add 10 μM of Rock inhibitor (Y27632) in the existing culture medium in the spinner flask. Incubate in an incubator (37° C.) for 1-2 hours before MACS.
Remove all media and wash with ice cold PBS. Then centrifuge at 300× g for 3 min.
Remove DPBS and resuspend with pre-warmed 1× TrypLE Select (Thermo 12563011) and incubate at 37° C. for 2 min and pipette up and down.
Incubate 2 min again. Carefully pipette up and down to separate single cells from cell aggregates. Wash with cold DPBS.

### Standard of Procedure for Magnetic Cell Separation (MACS) A. Magnetic Labeling

Collect all cells and count Cell number.
Centrifuge cell suspension at 300 x g for 3 min. Aspirate supernatant carefully.
Resuspend cell pellet with 1 mL buffer (Miltenui 130-091-221) and stain with the primary antibody (20 uL/10⁷ cells or 40 uL/10⁸ cells) (CD326-Microbead, Miltenui cat#130-061-101 and CD309-Biotin, Miltenui cat#130-093-603)
Mix well and incubate for 20 minutes on ice in the dark.
Wash cells to remove unbound primary antibody by adding 1-2 mL of buffer per 10⁷ cells and centrifuge at 300× g for 3-5 min.
Repeat washing step.
Aspirate supernatant carefully and resuspend cell pellet in 1 mL of buffer.
Add 20 uL of 2^{nd} antibody per 10⁷ total cells (Streptavidin Microbead, cat#130-048-102)
Mix well and incubate for 20 minutes on ice in the dark.
Wash cells by adding 1-2 mL of buffer and centrifuge at 300× g for 3 min.
Aspirate supernatant carefully
Repeat washing step
Resuspend up to 10⁷ cells in 5 mL of buffer.
Filter it through 70 μm cell strainer (Falcon, cat#352350).
Process to magnetic Separation. Use up to 100 million cells per column.

### B. Magnetic Separation

Place column (LD Column Cat# 130-042-901) in the magnetic Field of a suitable MACS separator.
Prepare column by rinsing with 2 mL of buffer
Apply cell suspension onto the column, about 10⁷ cells per column. Suitable collection tubes (15 mL tube with 1 mL of X-Vivo+10 μM Rock Inhibitor) to collect non-labelled cells (CD326- and CD309-).
Collect CD309 and CD326 negative cells that pass through and wash column with the appropriate amount of buffer (Perform column washing step by adding 1 mL buffer three times). * Only add new buffer when the column reservoir is empty.
After collection is done, count the cell number
Centrifuge at 300× g for 3 min and remove all supernatant carefully.
Resuspend cell pellet with CI-B (Chondro Induction) media (10 ng FGF₂, 10 ng IGF₁, 25 ng SHH, 50 ng BMP4, Rock Inhibitor 10 μM and Primocin). Apply to collagen membrane and incubate in 5% oxygen chamber. 10 mL of medium per membrane maximal density is 10 million per cm². Change medium daily for 7 days. Large scale separation can be done using CliniMax MACS Separator (Miltenui).
   Chondrogenic Induction (CI) (7 days).
POST MACS DAY (CI-B) in 96-well Membrane in Hypoxia Chamber (5% oxygen) add
X-Vivo
Rock Inhibitor (Y27632) 10 μM
FGF₂ 10 ng
SHH 25 ng
BMP4 50 ng
IGF 1 10 ng
Primocin 1:500

### Maintenance Media—up to day 40

Maintenance Medium is continued to be used in the same wells as CI medium. Use 10 mL of medium per membrane. Change medium every other day.
X-Vivo
IGF₁ 10 ng
FGF₂ 10 ng
LIF 50 ng
TFGβ1 10 ng
BMP4 3 ng
Primocin 1:500

### Freezing

Collagen Membrane with cells are cryopreserved in MesenCult ACF Freezing media (Stemcell Tech—05490) containing 10 uM Y27632 (Tocris 1254). Cells are frozen down with a controlled rate freezer, then are stored in liquid nitrogen vapor phase after 24 h in −80° C. freezer.

Reagent List and Vendor information

| Reagent | Company | Catalog # |
|---|---|---|
| Matrigel hESC Qualified Matrix | Corning | 354277 |
| mTeSR Plus | Stemcell | 05825 |
| | Technologies | |
| ReLeSR | Stemcell | 05873 |
| | Technologies | |
| Y27632 (Rock Inhibitor) | Tocris | 1254 |
| Recombinant Hunan WNT3a | R&D | 5036-WN-010 |
| Recombinant Human FGF2 | Peprotech | 100-18B |
| (FGF-Basic) | | |
| Recombinant Human Activin A | Peprotech | 120-14 |
| Recombinant Human Noggin | Peprotech | 120-10c |
| Recombinant Human BMP4 | Peprotech | 120-05ET |
| Recombinant Human SHH | Peprotech | 100-45 |
| Recombinant Human IGF1 | Peprotechi | 100-11 |
| Recombinans Human LIF | Peprotech | 300-05 |
| Recombinant Human TGF-b1 | Peprotech | AF-100-21C |
| X-Vivo | Lonza | 04-418Q |
| MesenCult ACF Freezing Media | Stemcell | 05490 |
| | Technologies | |
| Primocyn | In VivoGen | ant-pm-1 |

Cell numbers at different stages of cell differentiation

| Experiment | Pluripotent | Before MACS | After MACS (live) | Final Output |
|---|---|---|---|---|
| EXP14 | 1 BN cells in 3D | Total Cell # 745*10e6 | 200*10e6 | 10 MLN per cm² or 60 MLN per 6 cm² |
| | | Live Cell# 484.6*10e6 | | membranes (3 membranes) |

Proposed quality control testing of the intermediate/chondroprogenitors and Product/chondrocytes
Ensure the absence of bacteria, fungi, mycoplasma, viral adventitious agents, residual reprogramming vector and residual undifferentiated cells.
Control levels of endotoxin
Verify the identity and viability of the cells
Ensure that the cells are of normal karyotype
Ensure that the Drug Product cells are not tumorigenic
Assess the chondrogenic activity of Plurocart

Specifically, the absence of detectable levels of residual pluripotent and tumorigenic cells in the final product is determined by cell culturing and pluripotent gene expression quantitative PCR assays for the pluripotency genes Pou.5 and Lin28b. The baseline level of these genes should not exceed the levels of expression determined in fully specified human fetal chondrocytes at 8-9 weeks of development when no pluripotent cells are present in the joints.

Further, a quantitative PCR assay panel of cartilage genes is used to assess the chondrogenic activity of the product. The levels of COL2A1 gene 10-fold or higher and the levels of SOX9 gene 5-fold higher than the levels in undifferentiated ESI-017 is considered as acceptable release criteria.

Viability of the final product is assessed using quantitative CellToxTM Green (Promega CAT# G8741). Assessment of the viability is performed without digestion of the final product into single cells. Digestion of Plurocart cell sheet may lead to lower viability due in large part to the digestion step itself. The number of live cells is then calculated after measuring the amount of total and dead cells in a sample using a fluorescent plate reader. In parallel genomic DNA assay is carried out to determine the total number of cells in the tested membrane. At least 500,000 of viable cells per cm² is used as acceptable release criteria. Stability is assessed by performing viability testing of cryopreserved product every 3 month. Viability of the product should not decline below 500,000 of viable cells per cm² at any time of testing.

Scaled production and formulation optimization of hES-derived chondrocytes. Studies were performed to assess the long-term therapeutic potential of hESC-derived chondrocytes in a porcine model of focal articular cartilage injury. New and large-scale production methods were developed to produce articular cartilage-like chondrocytes from human PSCs. Cells generated using this technique are immature based on their transcriptional signature and expression of immature chondrocyte markers but can mature upon implantation in vivo, evidencing appropriate expression of superficial zone markers and lack of hypertrophy. For this study, procedures initially developed for H1 and H9 lines were redesigned to utilize the research grade hESC line ESI-01726. This specific line was selected because a cGMP version of this line is fully compliant with all current FDA regulations and can be advanced into human clinical trials without any regulatory restrictions.

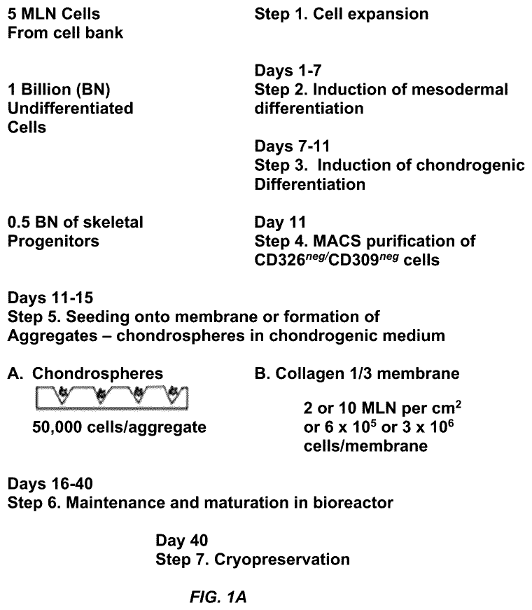

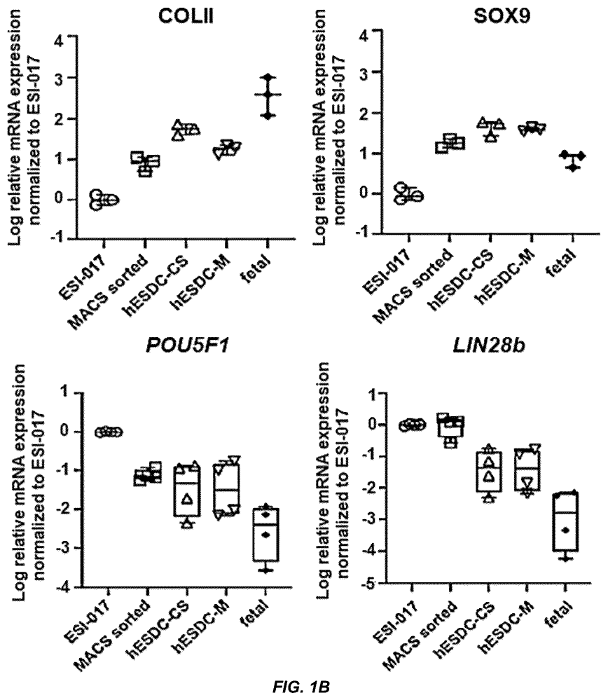

In order to generate sufficient numbers of clinical grade hESC-derived chondrocytes for cartilage defect repair, hESCs were first expanded in hESC-qualified Matrigel and induced into mesodermal differentiation (d1-7) followed by chondrogenic differentiation (d7-11; FIG. 1A). At d11, mesodermal skeletal progenitors were isolated using MACS to deplete for epithelial (undifferentiated and epidermal, EpCAM/CD326+) and cardiovascular mesodermal (KDR/CD309+) 27 cells (FIG. 1A).

During the optimization stage 2 different formulations of hESC-derived chondrocytes: chondrospheres (CS) and chondrocytes integrated onto a collagen I/III membrane previously approved for clinical use (Cartimaix; Matricel), were developed and tested. For chondrosphere production, a commercially available low attachment plate with a patterned floor designed was used to generate chondrospheres of uniform size and quality from d11 MACS-purified chondrogenic cells (FIG. 1A). In parallel, collagen membranes were sized to 6 mm (0.28 cm²) with a biopsy punch and seeded with purified skeletal progenitors isolated after MACS and transferred aseptically into the bioreactor. A continuous perfusion bioreactor system was used (FIG. 1A) for expansion and chondrocyte maturation for an additional 25 days to provide a stable microenvironment with precise control of gases, nutrients and physical parameters such as shear stress. ESI-017 cells responded to the established protocol by upregulating chondrogenic and downregulating pluripotency genes during the course of the manufacturing process (FIG. 1B).

Cryopreservation media (FIG. 7) was tested for each of these formulations to support the development of a universal, off-the-shelf potential therapy for articular cartilage defects. After optimization of cryopreservation, both chondrospheres and membranes were revived using MesencultTM ACF (FIG. 7). These batches routinely yielded dozens of chondrospheres containing ˜5×10⁴ cells, or low and high dose membranes containing 6×10⁵ or 3×10⁶ immature chondrocytes each, respectively.

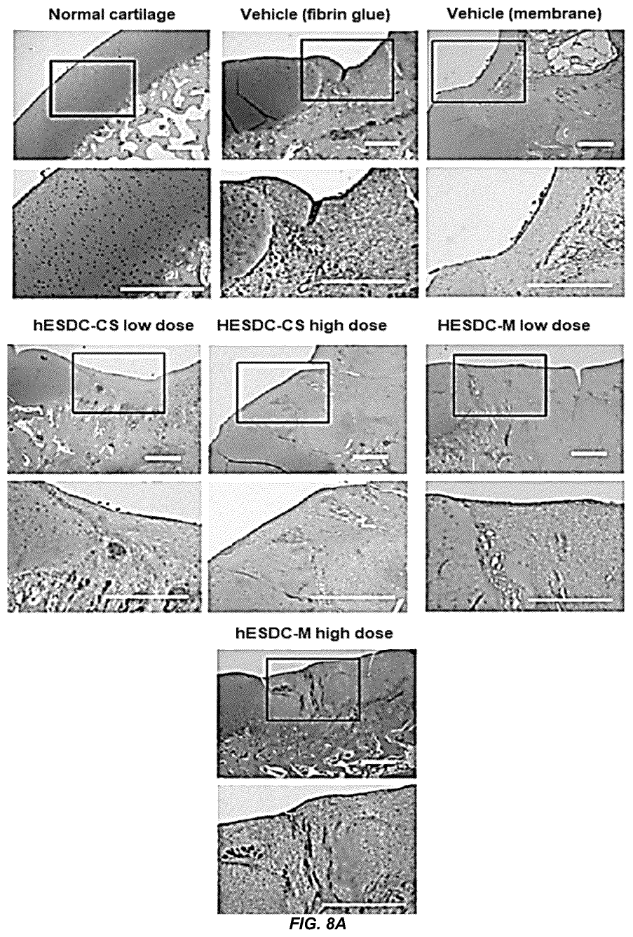

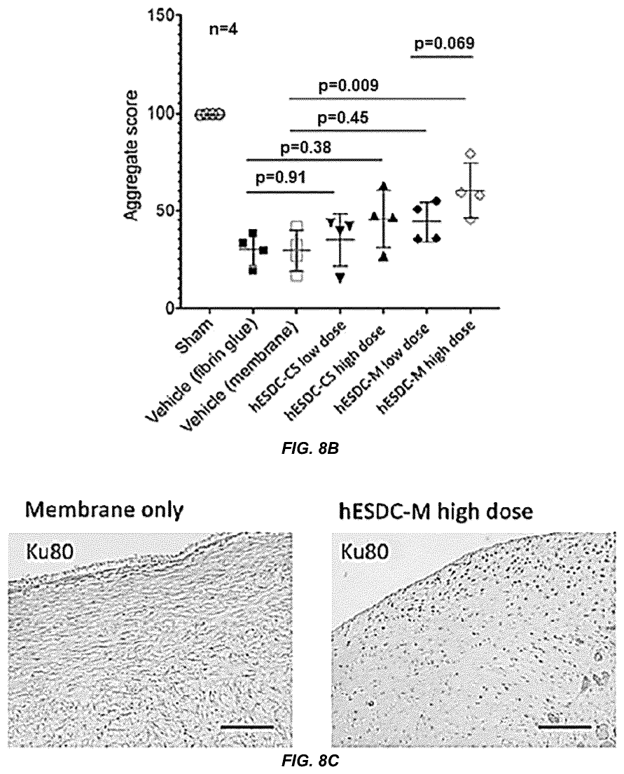

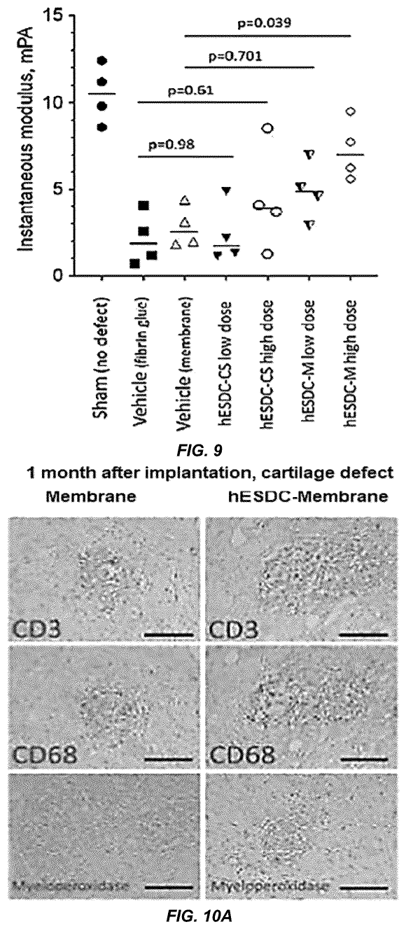

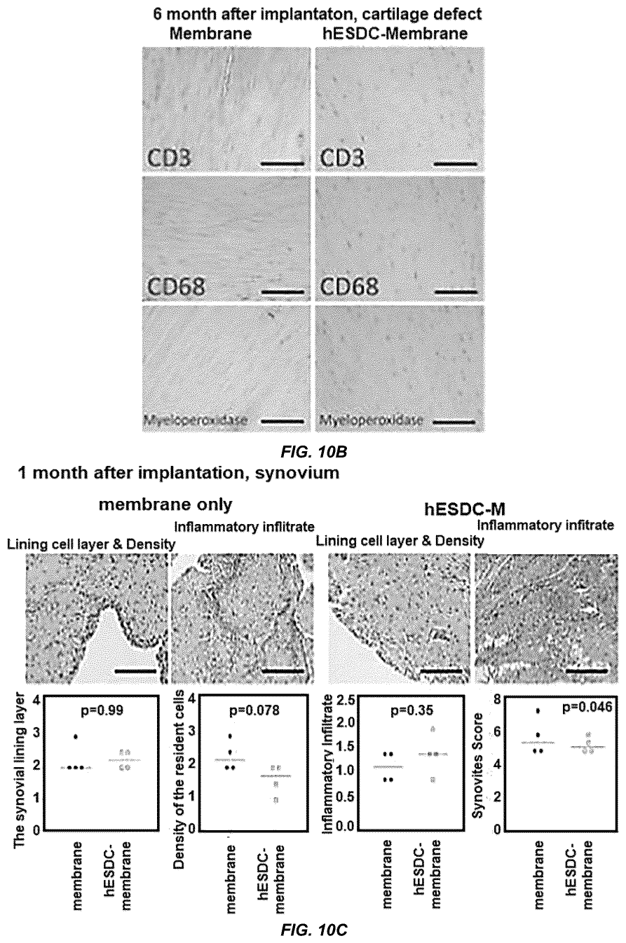

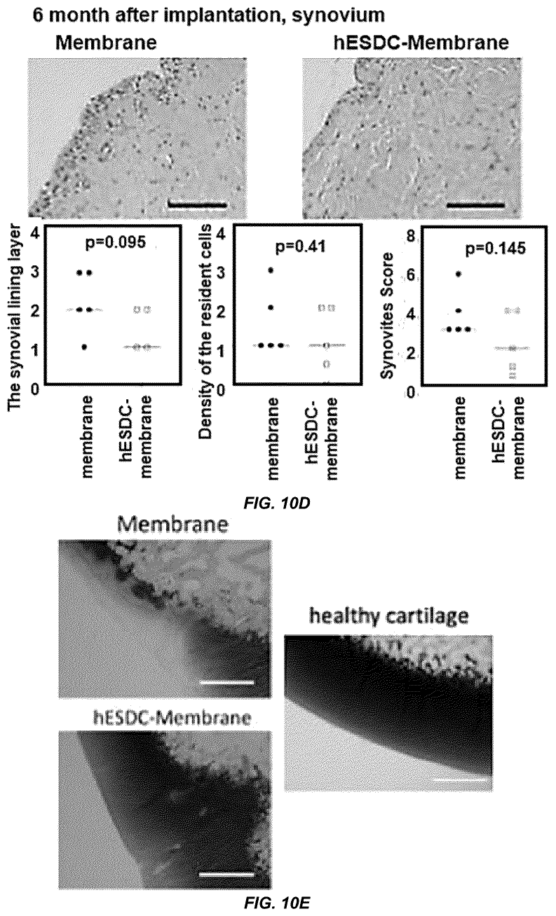

The ability of two doses of each formulation to support short-term repair of a focal defect in pig articular cartilage were compared (FIG. 8). Six millimeter full-thickness cartilage defects were created to the depth of the subchondral plate in the femoral condyle and low and high doses of hESC-derived chondrospheres or chondrocytes on membranes were implanted (empty membranes or glue alone were used as controls. One month later, injured areas were assessed for evidence of tissue integration, matrix production, fibrosis and presence of Ku80+ human cells; detailed biomechanical mapping was also conducted to determine biomechanical characteristics of the healing defects. These results (FIGS. 8 and 9) demonstrated that a high dose of chondrocytes embedded in collagen membranes was better maintained in the injury site and supported superior functional repair without eliciting a heightened immune response (FIG. 10). Based on these data, membrane-bound hESC-derived chondrocytes (hESDC-M) were used for further characterization and functional testing.

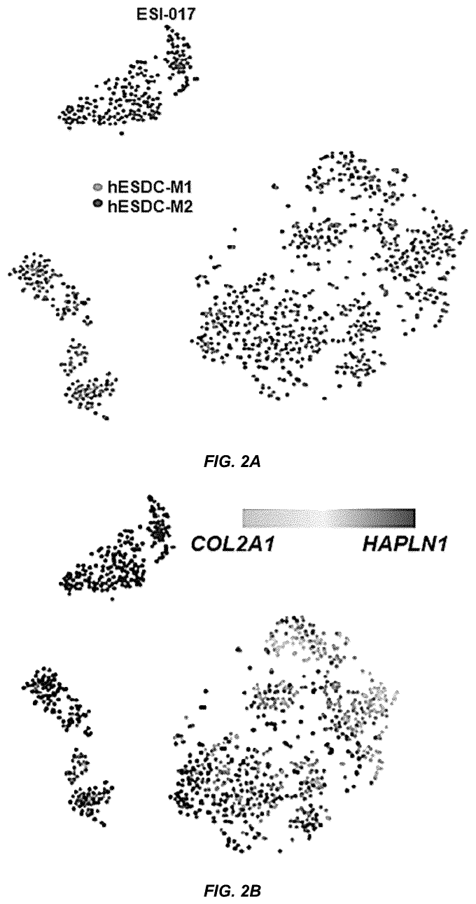

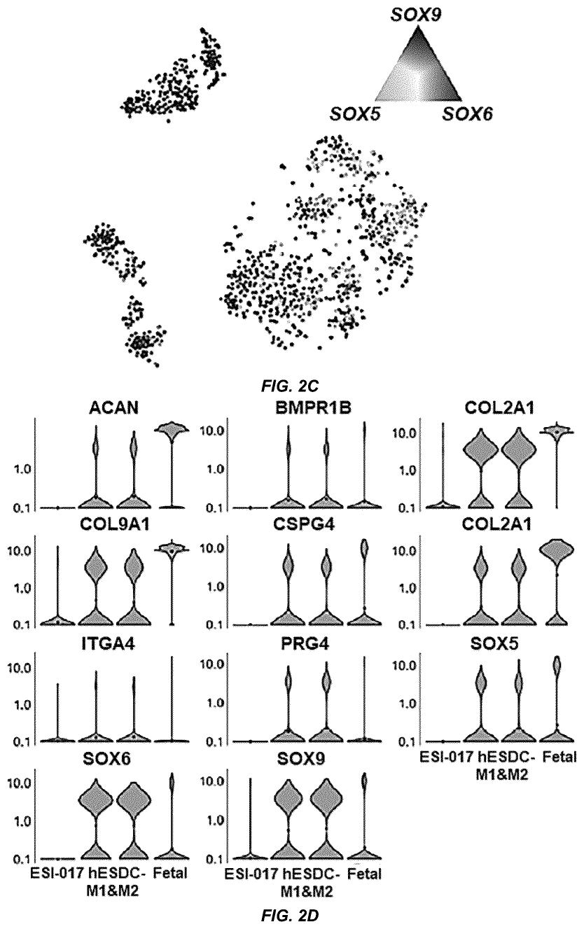

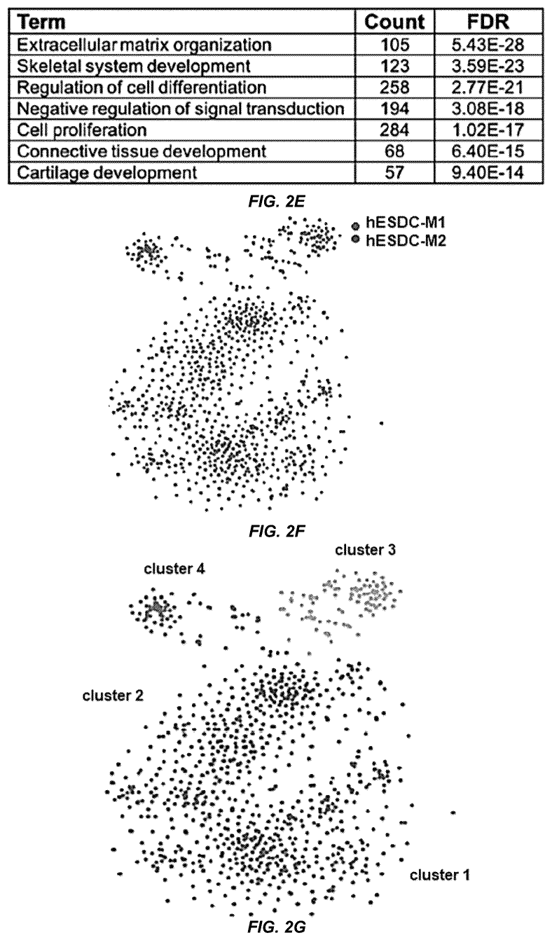

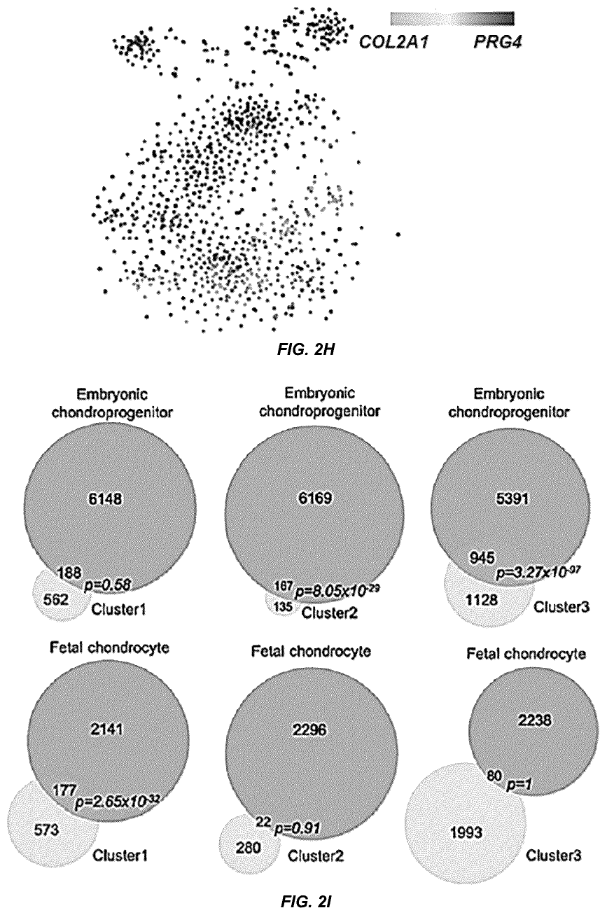

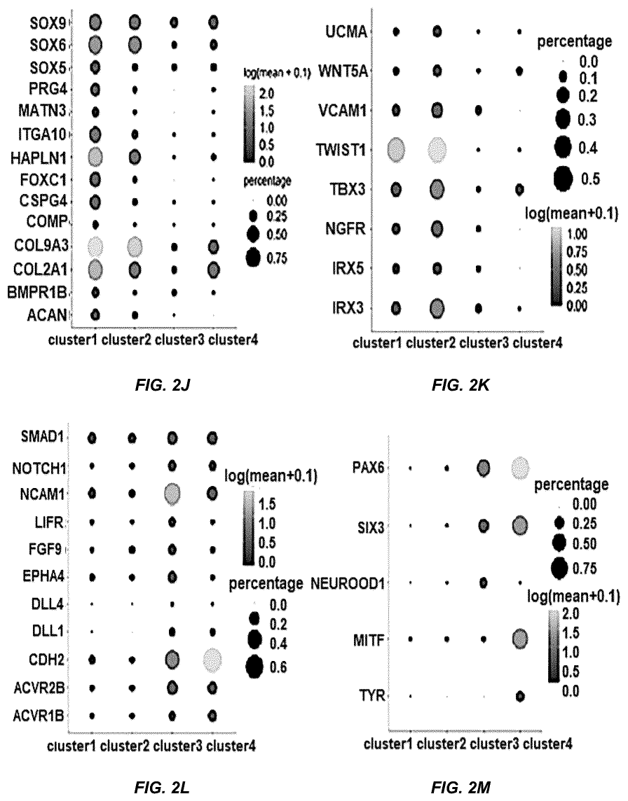

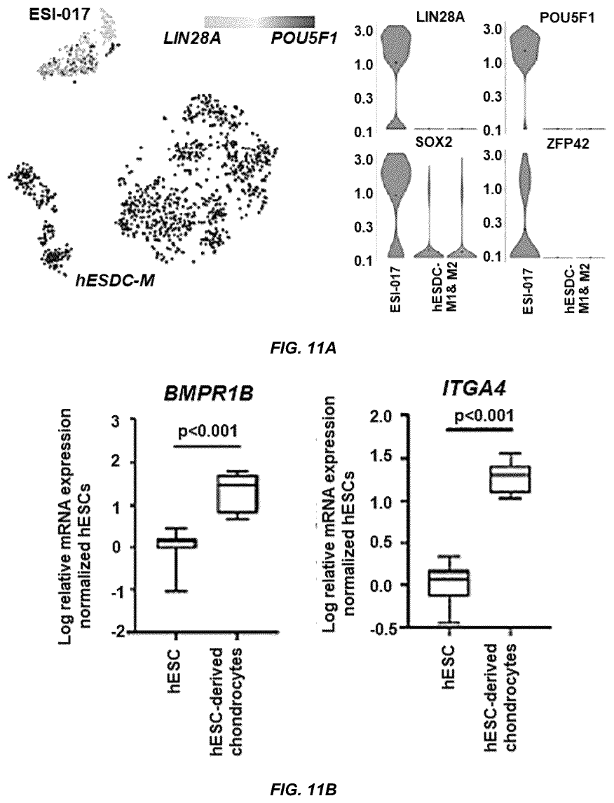

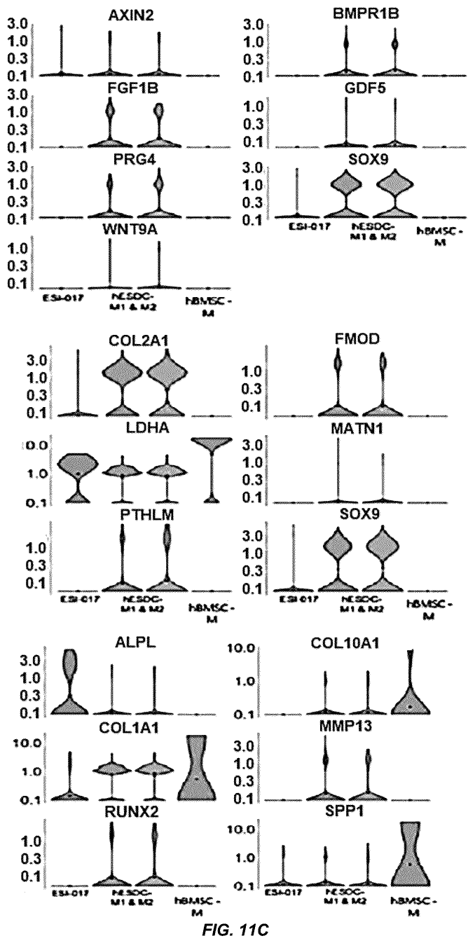

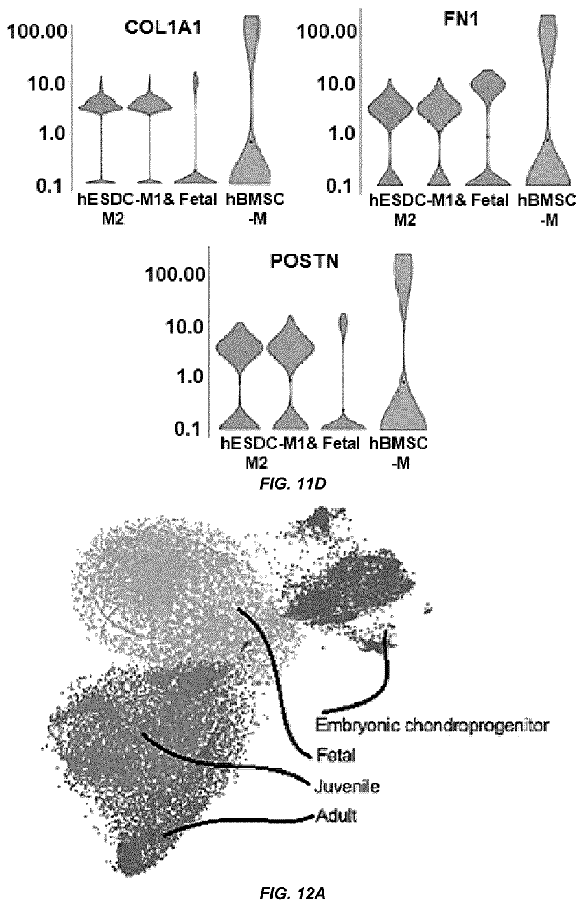

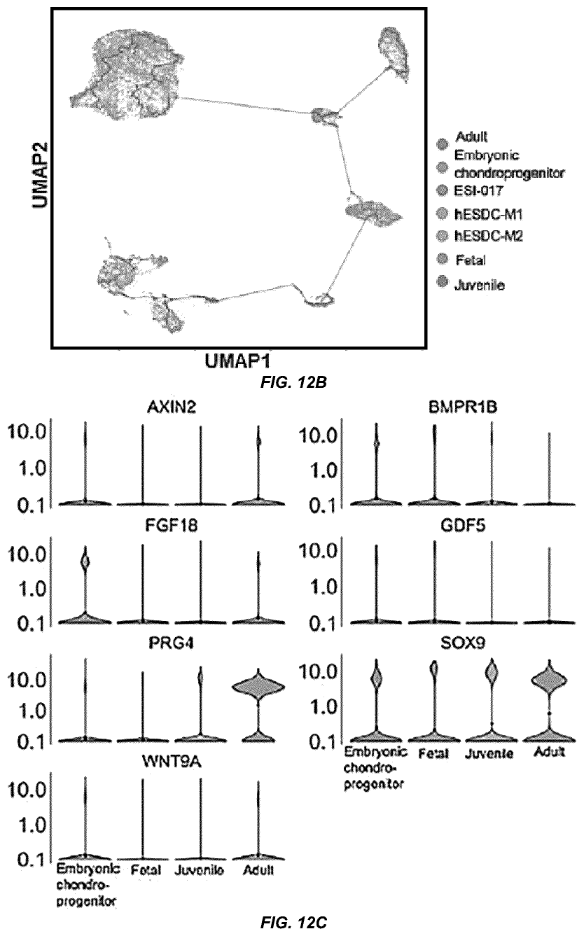

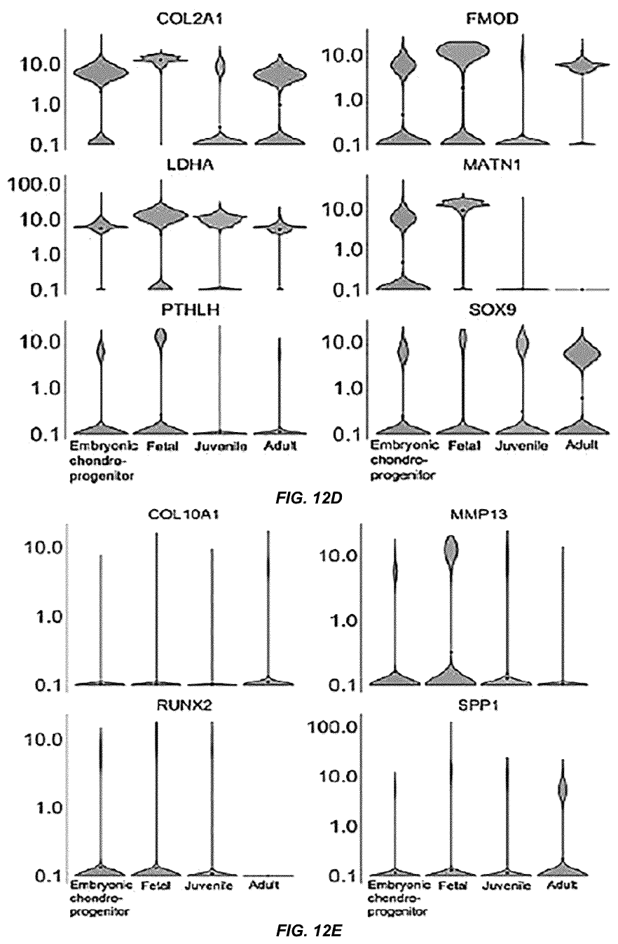

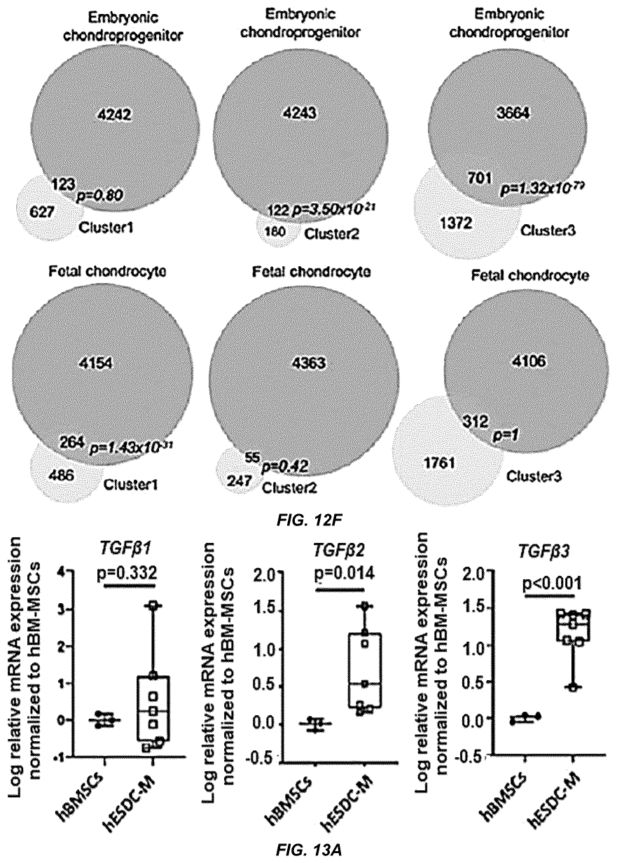

Characterization and developmental status of hESDC-M. To better understand the heterogeneity present in hESDC-M, single cell RNA-seq (scRNA-seq) was performed on cells isolated from two different batch production runs and compared them to undifferentiated ESI-017 cells (FIG. 2). Few to none of the cells demonstrated expression of pluripotency genes (POU5F1, LIN28A or ZFP42; FIG. 11A), suggesting limited potential for generation of teratomas upon transplantation in vivo. Both production runs contained similar cell types as shown by objective clustering (FIG. 2A), with the absolute majority of cells present being positive for chondrogenic markers including COL2A1, HAPLN1, and SOX5/6/9, (FIG. 2B-D). This was affirmed by gene ontology analysis of genes enriched in the hESDC-M (FDR<0.05, >2-fold change) as demonstrated by significant over-representation of genes related to cartilage development, matrix production and lineage commitment (FIG. 2E). Objective clustering of hESDC-M defined 4 subtypes of cells present on the membranes, potentially representing a continuum of chondroinduction and chondrogenesis and/or chondrogenic cells with different mesodermal origins; clusters 1 and 2 were the most enriched for COL2A1 and PRG4 (FIG. 2F-H). To define how these subtypes of hESDC-Ms compare with different stages of human ontogeny and human bone marrow stromal cells cultured on membranes (hBMSC-M), SCRNA-seq was performed at multiple stages of human chondrogenic ontogeny and on hBMSC-M (FIGS. 11 and 12). These data defined cluster 1 as the most mature, with highly significant overlap with genes enriched in fetal chondrocytes vs. embryonic chondroprogenitors, including genes encoding ECM proteins such as COL2A1, PRG4 and ACAN (FIG. 21-J; FIG. 12). Clusters 2 and 3 were more closely related to chondroprogenitors and chondroinductive cells present early during development, showing enrichment for genes involved in the generation of chondrogenic condensations and primitive mesoderm including TWIST132, NCAM1 and CDH233 (FIG. 2K-L; FIG. 12). These data were confirmed by comparison to previous bulk sequencing data generated at these stages (FIG. 12). Cluster 4 contained cells expressing COL2A1 and neural markers including PAX⁶ and MITF (FIG. 2M). A trajectory analysis of COL2+ cells was performed from each stage of human ontogeny and in vitro differentiation; these results placed hESDC-Ms between the embryonic and juvenile stages of human ontogeny (FIG. 12B). Conversely, hBMSC-M cultured under identical conditions to hESDC-M expressed genes associated with terminal chondrogenesis including COL10A1 and SPP1 (FIG. 11C-D). These data reveal that although the cells present on the membrane are heterogenous, the production process is reproducible and generates mostly immature chondrogenic and chondroinductive cells.

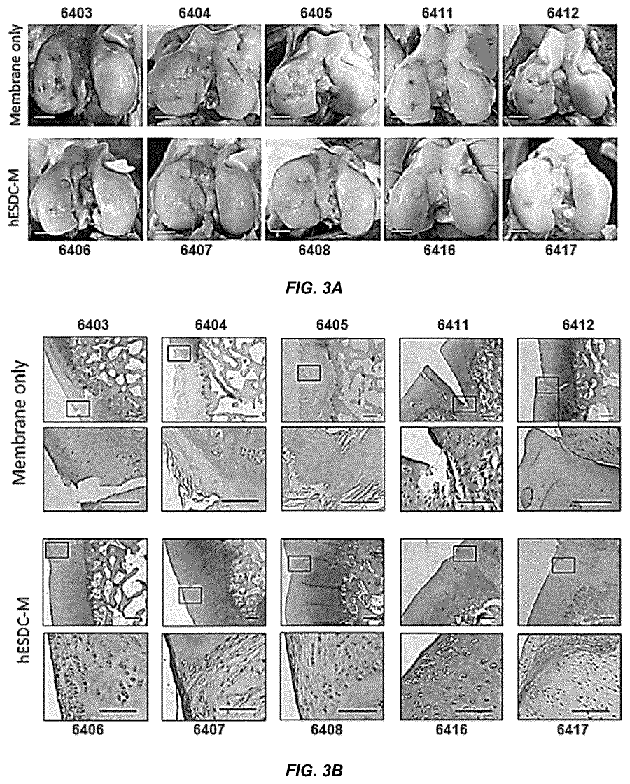

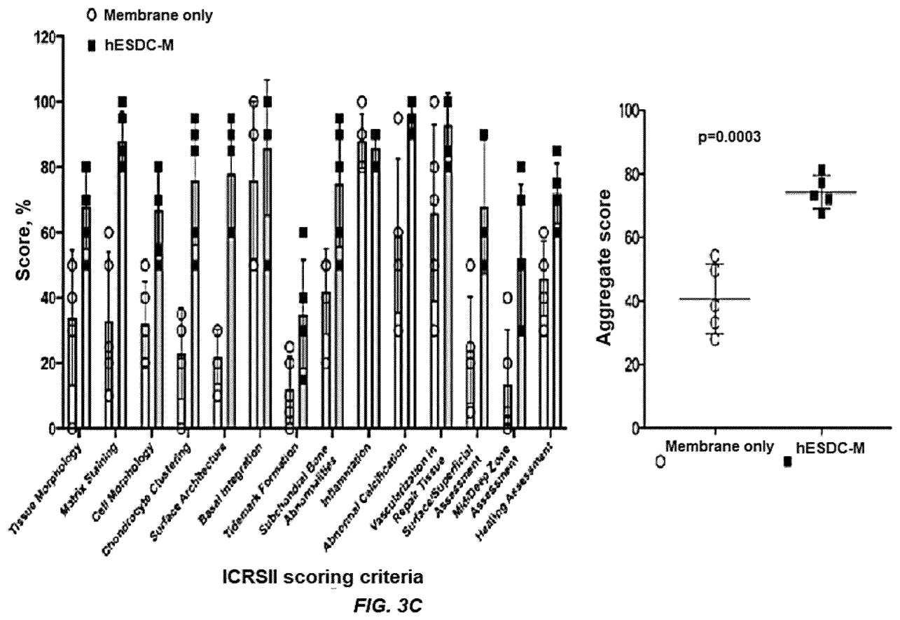

hESDC-M support long-term repair of articular cartilage in pigs. In order to assess the therapeutic potential of hESDC-M, a long-term clinically relevant experiment was performed in which either membranes alone or membranes with cells were implanted into pig articular cartilage defects and assessed 6 months later. Pigs in each group (n=5) had 2, 6 mm full-thickness cartilage defects created with an average of 7 mm apart in their femoral condyles within the load bearing areas and were treated with either membranes alone or hESDC-M; two animals were used as sham controls with no cartilage defects generated. Cell implants were thawed and washed in fresh X-Vivo media approximately 1 h prior to implantation, applied to the defects, and were fixed in place with fibrin glue. After 6 months, pigs were euthanized and cartilage assayed using morphological, histological and biomechanical methods. Defects from all pigs transplanted with cells uniformly evidenced substantially less degeneration in and around the injury site (FIG. 3A). Sham operated animals showed no noticeable morphological or biomechanical differences with non-operated knees. At the microscopic level, defects treated with cells contained neocartilage with more proteoglycan deposition and better integration of the new cartilage tissue with the non-injured surrounding matrix (FIG. 3B, FIG. 10E). To quantify the extent of regeneration provided by cells, all 10 defects per group were scored by 2 blinded observers using the International Cartilage Repair Society (ICRS) II histological assessment system (FIG. 3C). This scoring system grades 14 criteria relevant to cartilage repair and provides a comprehensive view of the utility of potential treatment. Scores from each observer for each defect were averaged to provide a composite for each criterion. These data showed significantly better outcomes for defects treated with hESDC-M (FIG. 3C). This was confirmed by synovitis scoring and staining for inflammatory infiltrates, which showed no difference between animals implanted with empty membranes vs. those receiving hESDC-M, further supporting the low immunogenicity of hESDC-M (FIG. 10). Moreover, morphological signs of synovitis were more prominent in pigs treated with membranes only, likely reflecting progression of degenerative joint disease in this group; however, this difference did not reach statistically significant values (FIG. 10D). Histological analysis of the subchondral bone showed no major differences between the groups.

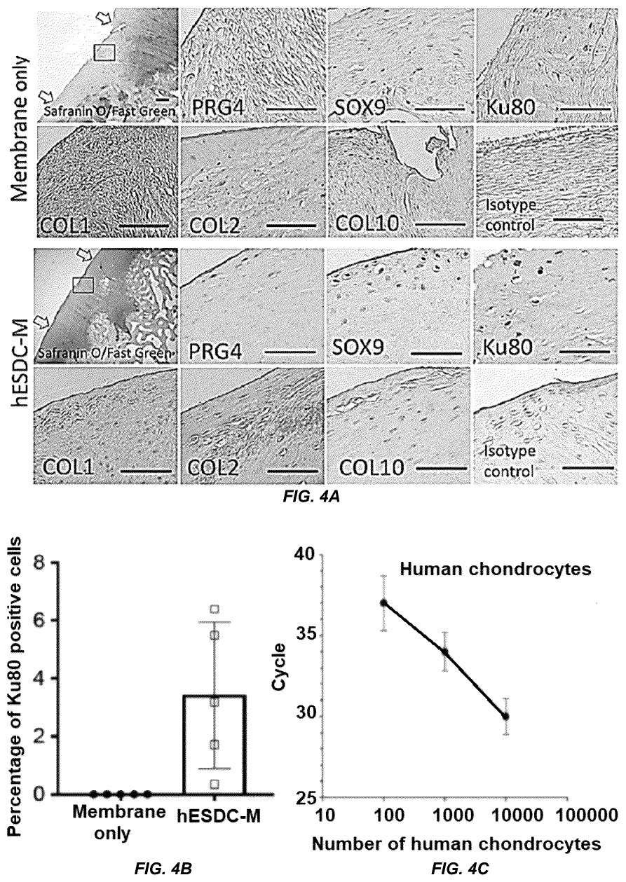

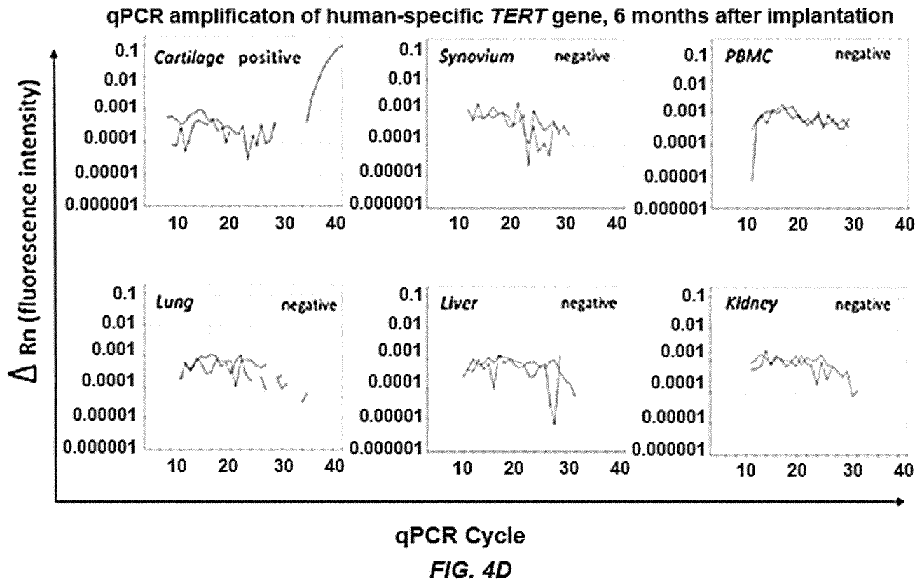

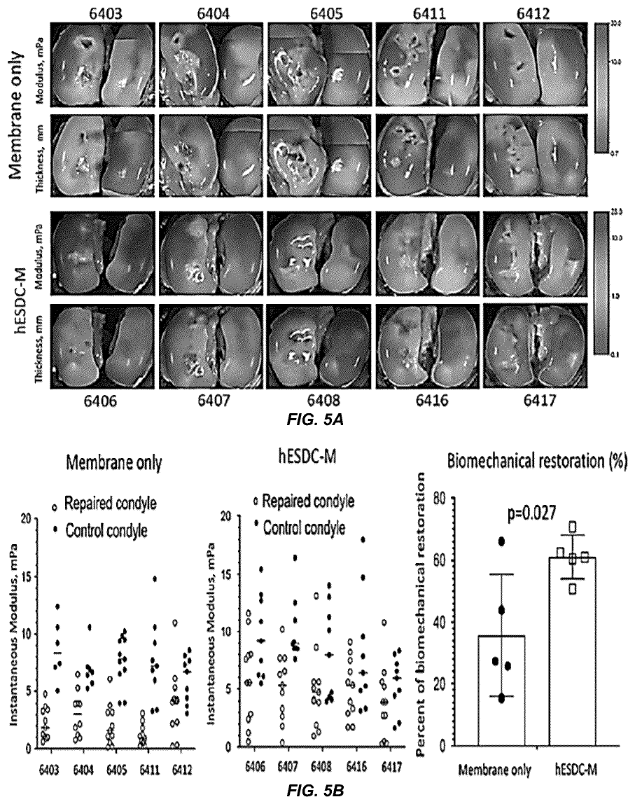

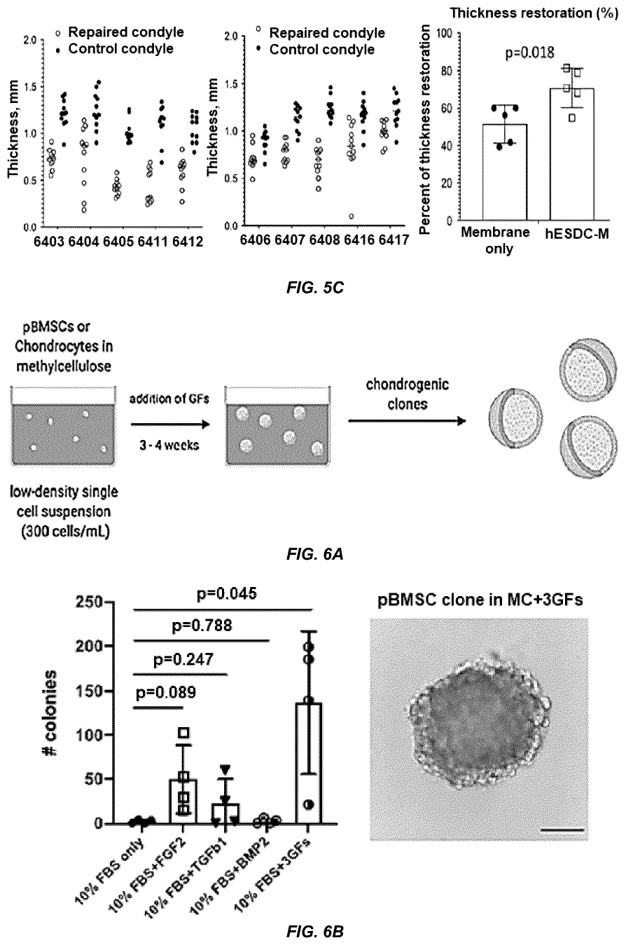

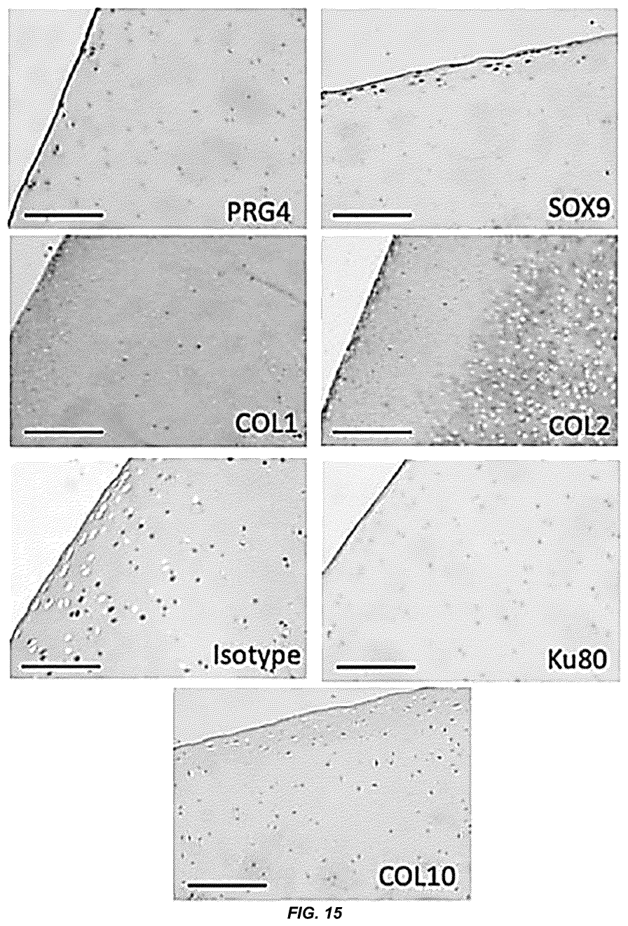

At the molecular level, cartilage in the defects treated with cells more closely resembled the surrounding tissue. In membrane only defects, much of the new tissue was inferior as evidenced by collagen 1 and collagen X staining coupled with substantially reduced proteoglycan content (FIG. 4A). In contrast, defects transplanted with hESDC-M evidenced appropriate stratification of the neocartilage as demonstrated by superficial production of lubricin (PRG4) and localization of SOX9⁺ cells (FIG. 4A). Moreover, substantial production of collagen II in the transitional zone was primarily observed in defects treated with cells, showing similar collagen deposition compared to normal pig cartilage (FIG. 15). Notably, even after 6 months following implantation, small clusters of Ku80⁺ human cells were identifiable in all treated animals (FIG. 4B). With the safety profile of hES-derived chondrocytes in mind, the biodistribution of human cells was examined by using a sensitive PCR-based assay to detect human telomerase (TERT) in cartilage, synovium, peripheral blood and major organs (FIG. 4C-D). While the presence of human cells in the repaired articular cartilage was confirmed and represented roughly 4% of total cells (FIG. 4B), levels of human DNA in all other tissues analyzed, including synovium, was below the threshold of detection, indicating that transplanted cells do not leave the defect following implantation after 6 months.

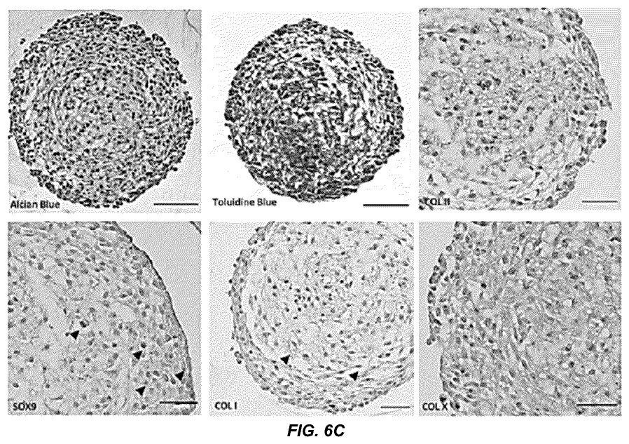

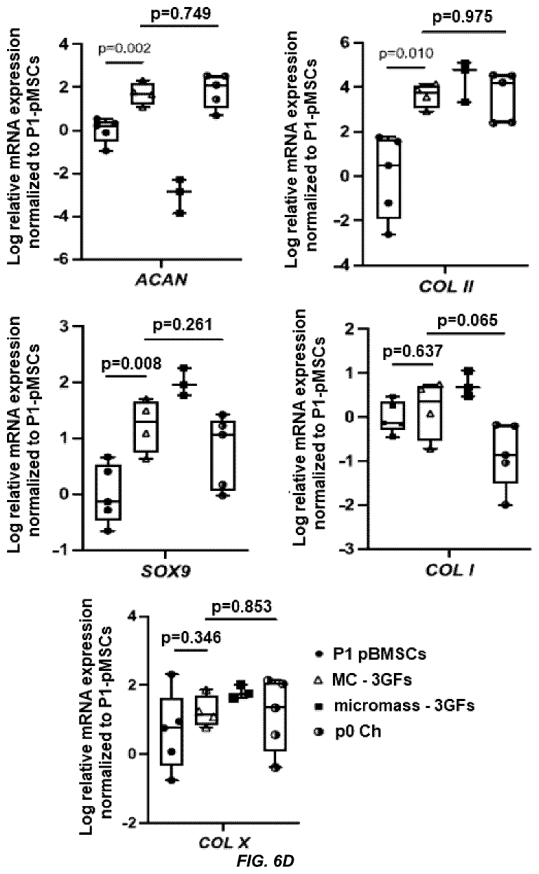

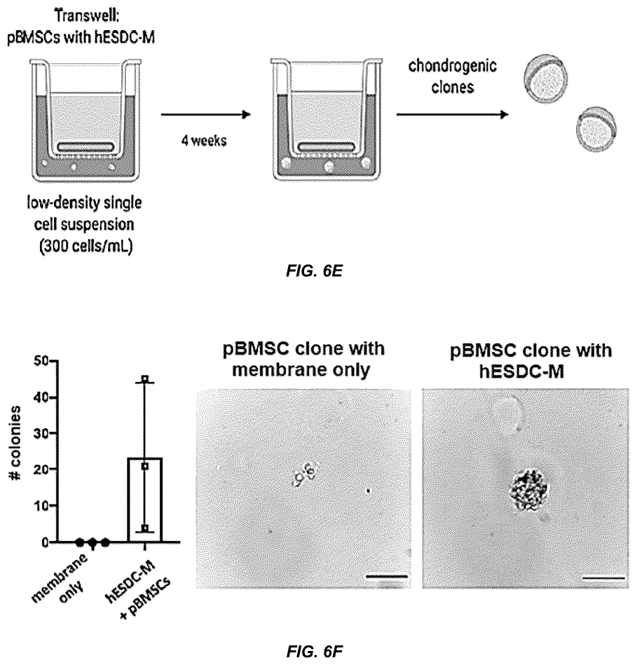

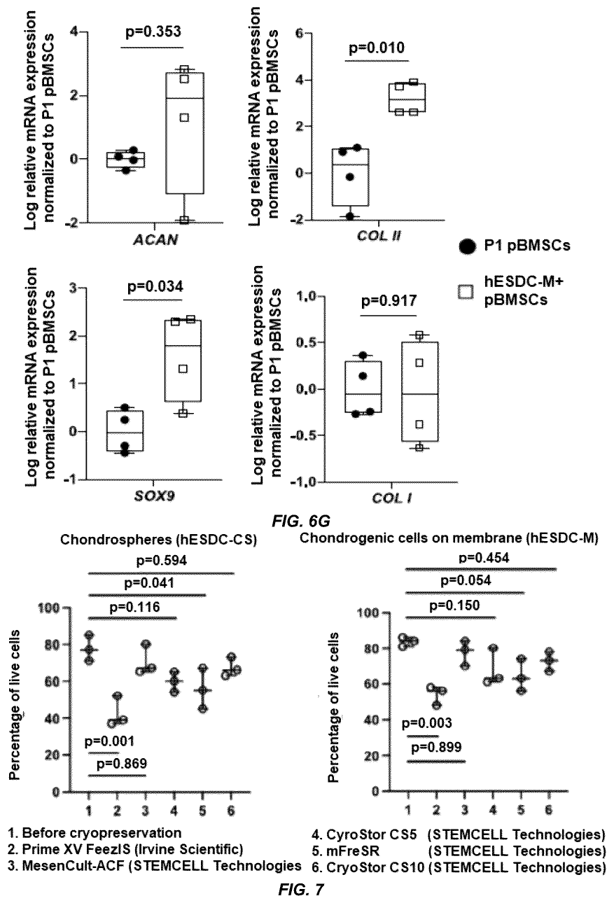

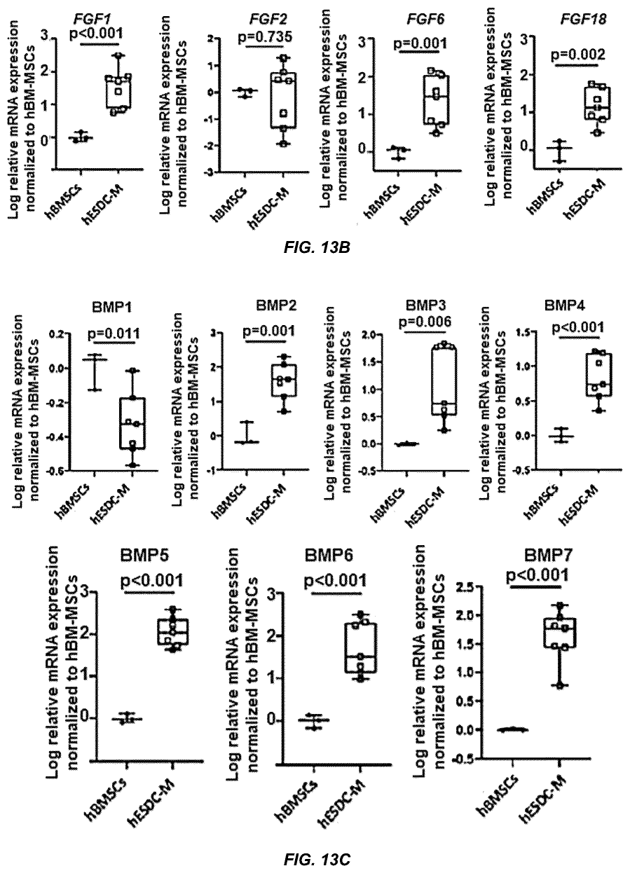

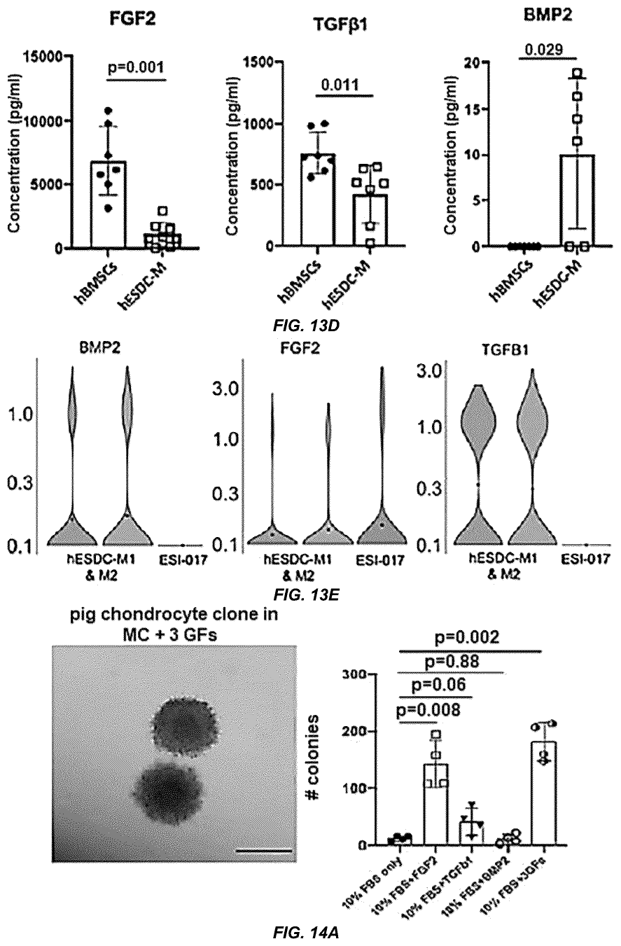

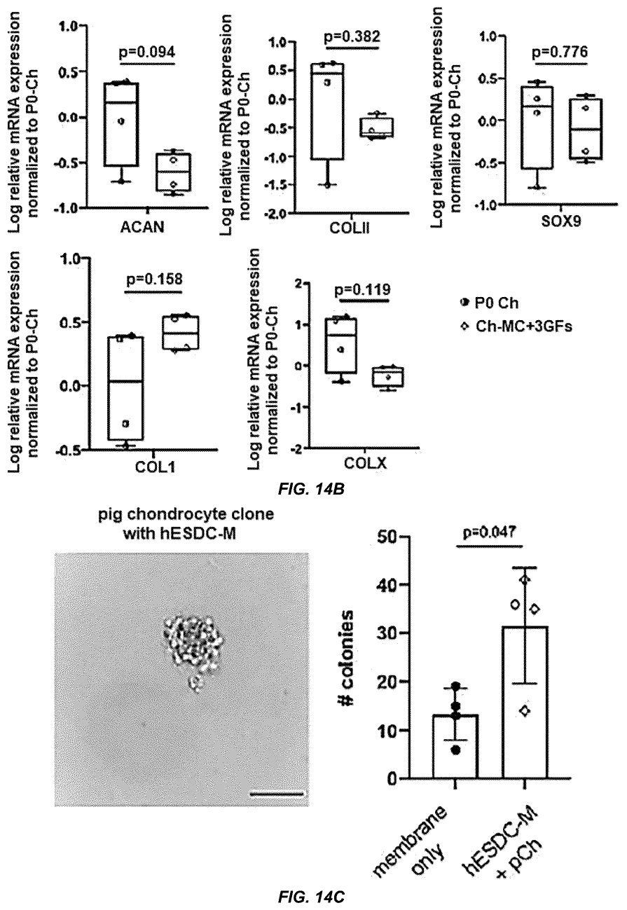

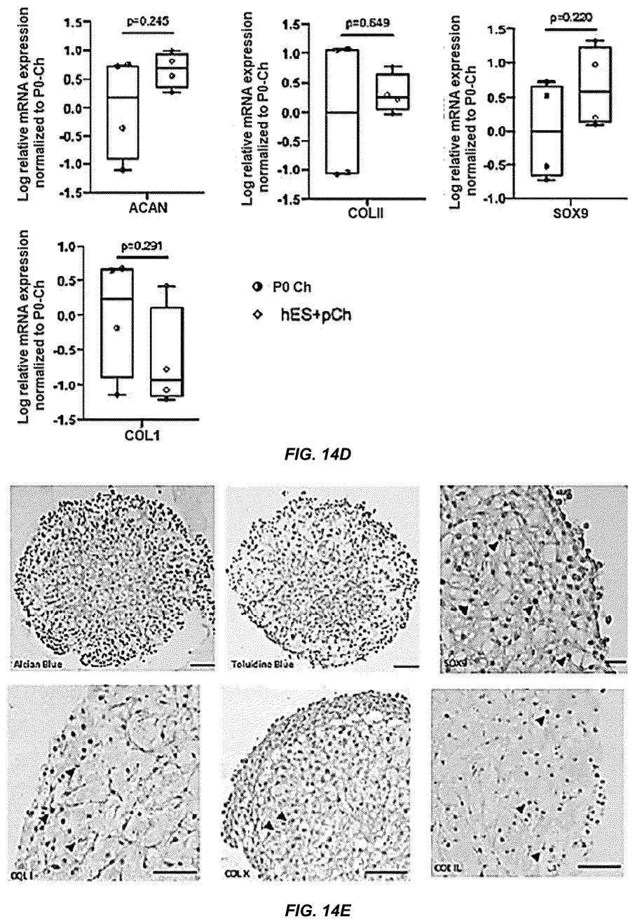

hESDC-M secrete chondroinductive factors that induce chondrogenesis from porcine BMSCs. Given that the majority of hyaline-like neocartilage was contributed by pig cells, the chondroinductive, paracrine effects of hESDC-M on pig BMSCs and chondrocytes were assessed (FIGS. 6 and 14), the cells likely responsible for generating neocartilage in the pig model. A methylcellulose (MC)-based culture method was used to assess both clonality at a single cell level and capacity of pig BMSCs to undergo chondrogenesis in vitro (FIG. 6A). Growth factors of the TGF-β, FGF and BMP families are known to be chondroinductive during development and following injury. Upon this basis, three growth factors were selected (FGF-2, BMP-2, and TGF-β1, “3GFs”) produced by hESDC-M (FIG. 13) and added them to the MC-based media, showing that a combination of all 3 growth factors yielded the most clones (FIG. 6B). Notably, BMP-2 was not secreted by hBMSCs (FIG. 13D), suggesting that endogenously activated BMSCs may not produce sufficient chondroinductive factors to generate hyaline-like neocartilage as was observed with hESDC-M. These colonies produced proteoglycans and other chondrogenic markers comparable to native articular chondrocytes cultured in the same method (FIG. 6C and 13E). Chondrogenic gene expression similar to that of native pig articular chondrocytes was evident when comparing BMSCs cultured in MC with 3GFs, with substantial induction of chondrogenic genes versus starting BMSCs (FIG. 6D). Moreover, culture in MC+3GFs yielded chondrogenic gene expression similar to micromass culture of pig BMSCs+3GFs, the standard method for generating chondrocytes from BMSCs in vitro. To assess whether paracrine factors produced by hESDC-M could promote chondrogenesis from pig BMSCs, a co-culture system with Transwell inserts and MC (FIG. 6E) was used. After 4 weeks of co-culture, clonality and chondrogenesis showed the same trends as BMSCs cultured with all 3GFs (FIG. 6F-G); expression of collagen X was undetectable. Importantly, hESDC-M also supported clonal chondrogenesis from pig chondrocytes (FIG. 14), suggesting two possible cellular sources for neocartilage following hESDC-M implantation. These data indicate secreted proteins such as BMP-2 produced by hESDC-M can promote induction of articular-like chondrogenesis from BMSCs, implying that the paracrine factors supplied are crucial to the generation of functionally superior neocartilage.

It is understood that various modifications may be made without departing from the spirit and scope of this disclosure. Accordingly, other embodiments are within the scope of the following claims.

1. A process to produce highly viable, engraftable human chondrocytes from pluripotent stem cells, comprising:
culturing stem cells on laminin coated tissue culture vessel(s) comprising a serum-free, stabilized cell culture medium for pluripotent stem cells;
transferring the stem cells to a 3D condition/spin culture system and culturing the stem cells in a medium for 5 days or more, wherein the medium comprises a serum-free, stabilized cell culture medium for pluripotent stem cells, and a ROCK inhibitor;
differentiating the stem cells to chondrocyte-like cells by culturing the stem cells under the following conditions:
(a) culturing the stem cells in a mesoderm induction differentiation medium for 3 days or more, wherein the mesoderm induction differentiation medium comprises a chemically defined, serum-free hematopoietic cell medium, ROCK inhibitor, Wnt3a, Activin A, and FGF2;
(b) culturing the stem cells in a MI-B differentiation medium for 3 days or more, wherein the MI-B differentiation medium comprises a chemically defined, serum-free hematopoietic cell medium, Wnt3a, Noggin, and FGF2;
(c) culturing the stem cells in chondrogenic induction differentiation medium A for 3 days or more, wherein the chondrogenic differentiation medium comprises a chemically defined, serum-free hematopoietic cell medium, FGF₂, and BMP4;
sorting the differentiated stem cells by using magnetic cell separation to identify CD309 and CD326 negative cells, which are collected;
culturing the CD309 and CD326 negative cells in chondrogenic induction differentiation medium B for seven days or more to form chondrocytes, wherein chondrogenic induction differentiation medium B comprises a chemically defined, serum-free hematopoietic cell medium, FGF2, IGF1, SHH, BMP4, ROCK inhibitor, and antimicrobials;
culturing the chondrocytes in maintenance media for 7 days or more; wherein the maintenance media comprises a chemically defined, serum-free hematopoietic cell medium, FGF2, IGF1, LIF, TGF-β1, BMP4, and antimicrobials;
optionally, treating the chondrocytes with an agent or compound that enhances anabolism, extracellular matrix secretion, promotes cell survival and or proliferation, reduces catabolismand/or reduces degeneration of extracellular matrix in chondrocytes; and
optionally, cryopreserving the chondrocytes with collagen membranes in a defined, serum-free, and animal component-free freezing medium that comprises a ROCK inhibitor.

2. The process of claim 1, wherein the stem cells are human embryonic stem cells or induced pluripotent stem cells (iPSCs).

3-4. (canceled).

5. The process of claim 1, wherein the serum-free, stabilized cell culture medium for pluripotent stem cells is mTeSR medium.

6. The process of claim 1, wherein the ROCK inhibitor is Y-27632.

7. (canceled).

8. The process of claim 1, wherein the a chemically defined, serum-free hematopoietic cell medium is X-Vivo medium.

9. The process of claim 1, wherein the magnetic cell separation utilizes antibodies to CD326 and CD309 in a biotin/streptavidin pulldown system.

10. A method of producing a cartilage composition comprising, inducing differentiation of a stem cell culture comprising:
(a) culturing stem cell aggregates in mesoderm induction media A (MI-A) for 2-5 days, wherein the MI-A media comprises a chemically defined, serum-free hematopoietic cell medium containing Rock Inhibitor (10 uM), Wnt3a (10 ng/ml), bFGF (10 ng/ml) and activin A (10 ng/ml);
(b) replacing the media of (a) with mesoderm induction media B (MI-B) and culturing the cell aggregates for 2-5 days, wherein the MI-B media comprises a chemically defined, serum-free hematopoietic cell medium containing Wnt3a (10 ng/ml), bFGF (10 ng/ml) and Noggin (50 ng/ml); inducing chondrocyte induction comprising:
(1) replacing the media of (b) with chondrocyte induction media A (CI-A) and culturing the cell aggregates for 2-5 days, wherein the CI-A media comprises a chemically defined, serum-free hematopoietic cell medium containing 10 ug/ml bFGF and 50 ug/ml BMP4;
(2) culturing CD309 and CD326 negative cells from (1) in chondrogenic induction differentiation media B (CI-B) for 1-7 days to form chondrocytes, wherein CI-B media comprises a chemically defined, serum-free hematopoietic cell medium containing 10 uM Rock inhibitor, 10 ng/ml IGF-1, 10 ng/ml bFGF, 50 ng/ml BMP4 and 25 ng/ml SHH, (i) seeding the cells obtained in (2) on a collagen membrane and culturing with maintenance media, wherein the maintenance media comprises a chemically defined, serum-free hematopoietic cell medium containing 10 ng/ml IGF-1, 10 ng/ml bFGF, 50 ng/ml LIF, 10 ng/ml TGFb1 and 1 ng/ml BMP4, wherein the media is changed as needed,
thereby obtaining a cartilage composition.

11. The method of claim 10, wherein the stem cell culture comprises human embryonic stem cells or induced pluripotent stem cells (iPSCs).

12. (canceled).

13. The method of claim 10, wherein the ROCK inhibitor is Y-27632.

14. The method of claim 10, wherein the chemically defined, serum-free hematopoietic cell medium is X-Vivo medium.

15. The process of claim 1, wherein the chondrocytes are treated with an agent or compound that enhances anabolism, extracellular matrix secretion, promotes cell survival and or proliferation, reduces catabolismand/or reduces degeneration of extracellular matrix in chondrocytes.

16-20. (canceled).

22. A method comprising engrafting chondrocytes made by the process of claim 1 into a subject in need thereof.

23. The method of claim 22, wherein the chondrocytes engrafted are derived from iPSCS made from the subject's cells.

24. A collagen composition comprising cells obtained by the method of claim 1.

25. The collagen composition of claim 24, wherein the collagen composition is a collagen membrane.

26. The collagen composition of claim 24, wherein the cells express COL2A1, SOX5/6/9, and ACAN.

27. The collagen composition of claim 26, wherein the cells further express BMPR1B and ITGA4.

28. The collagen composition of claim 26 or 27, wherein the cells further express PRG4 and HAPLN1.

29. A population of isolated cells obtained by the method of claim 1.

30. The population of isolated cells of claim 29, wherein the cells express COL2A1, SOX5/6/9, and ACAN.

31. The population of isolated cells of claim 30, wherein the cells further express BMPR1B and ITGA4.

32. The population of isolated cells of claim 29 or 30, wherein the cells further express PRG4 and HAPLN1.

33. A population of isolated cells, wherein the cells can differentiate into chondrocytes and wherein the cells express the markers COL2A1, SOX5/6/9, and ACAN.

34. The population of isolated cells of claim 33, wherein the cells further express BMPR1B and ITGA4.

35. The population of isolated cells of claim 33 or 34, wherein the cells further express PRG4 and HAPLN1.
